(19) United States
(12) Patent Application Publication
(10) Pub. No.: US 2003/0039976 A1
(43) Pub. Date: Feb. 27, 2003

(54) Methods for base counting

(76) Inventor: Lawrence A. Haff, Westborough, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/929,507

(22) Filed: Aug. 14, 2001

(51) Int. Cl. C12Q 1/68; C12P 19/34

(52) U.S. Cl. 435/6; 435/91.2

(57) ABSTRACT

Methods are provided for determining polynucleotide sequence information using mass-modified bases incorporated into amplification products. A sample including a target nucleic acid is amplified in the presence of a mass-modified nucleobase to produce an amplified product incorporating the mass-modified nucleobase. The mass of one strand of the amplified product is compared with the mass of one strand of a reference nucleic acid.

### FIELD OF THE INVENTION

The present invention relates to methods for determining sequence information of a polynucleotide. More specifically, the invention relates to methods for determining sequence information of polynucleotides using mass-modified bases.

### BACKGROUND OF THE INVENTION

It has become increasingly valuable to be able to rapidly and inexpensively identify variations from a normal genome, for example, a normal human genome. Often a single base change from a species'“normal” genome can have dramatic effects on the phenotype of an individual organism. Additionally, other mutations, including deletions, insertions, and duplications, can affect the phenotype of an organism. The challenge has been to find rapid, inexpensive methods to determine whether a nucleic acid contains such a changed sequence.

### SUMMARY OF THE INVENTION

The present invention provides methods for determining polynucleotide sequence information using a mass-modified nucleobase. Methods of the invention provide a straight-forward, inexpensive way to detect mutations in a sequence such as single nucleotide polymorphisms (“SNP's”), insertions, deletions, and length polymorphisms.

In one embodiment, a first sample of the target nucleic acid is amplified in the presence of three unmodified nucleobases (for example, dATP, dCTP, and dGTP) and one mass-modified nucleobase (for example, *dUTP, where the asterisk indicates a mass-modified base) to produce an amplified product which incorporates the mass-modified nucleobase. The mass-modified nucleobase can have a mass that is more than about 27 atomic mass units (“amu”) greater than the mass of the corresponding unmodified nucleobase. A second sample containing a target nucleic acid is amplified with four types of unmodified nucleobases (for example, dATP, dCTP, dGTP, and dUTP) to produce a reference nucleic acid. Subsequently, the masses of at least one strand of each of the amplified product and the reference nucleic acid are compared. The mass difference, if any, between at least one strand of each of the reference nucleic acid (without mass-modified nucleobases) and the amplified product (incorporating mass-modified nucleobase(s)) is divided by the mass difference between the unmodified nucleobase (for example, dUTP) and the mass-modified nucleobase (for example, *dUTP) to determine the number of mass-modified nucleobases of a given type incorporated in at least one strand of the amplified product (in this case, UTP). Ultimately, based on base pairing rules, the number of bases of a given type in the target nucleic acid is determined.

Base changes in the sequence of the target nucleic acid alter the number of bases of a given type in the target nucleic acid from those expected in a known normal sequence. Thus, a single base change (for example, a SNP) changes the identity of a single base in the target sequence and the number of the mass-modified nucleobases of a given type in an amplification product. Insertions, deletions, repeats, and other polymorphisms can alter the labeled base composition by more than one base. These changes also can be detected.

In another embodiment, prior to comparing the masses of at least one strand of each of the amplified product and the reference nucleic acid to detect an increase in mass, if any, a segment of nucleic acid is removed from the reference nucleic acid (without mass-modified nucleobases) and the amplified product (incorporating mass-modified nucleobase(s)). For example, if the polymerase chain reaction (“PCR”) is used to amplify the target nucleic acid, the sequence corresponding to the amplification primers can be removed. Because the removed segment is the same in both amplification products, the masses of shortened versions of one strand each of the reference nucleic acid and the amplified product are compared to determine the number of mass-modified nucleobases incorporated in one strand of the shortened amplified product.

In another embodiment, a first sample containing a target nucleic acid is amplified in the presence of three unmodified nucleobases (for example, dATP, dCTP, and dGTP) and one mass-modified nucleobase (for example, *dUTP) to produce an amplified product. A second nucleic acid sample is amplified in the presence of the same three unmodified nucleobases and one mass-modified nucleobase to produce a reference nucleic acid. The masses of one strand of each of the amplified product and the reference nucleic acid are compared. The mass difference, if any, between the amplified product and the reference nucleic acid is compared to determine whether the two amplification products have a different base composition for a base of a given type (in this case, uridine residues). Accordingly, the identity of a base responsible for a base composition difference, if there is any, between the amplified product and the reference nucleic acid can be determined.

In the above-described embodiment, the second amplification step need not occur, as the known mass of a reference nucleic acid can be used. Accordingly, only one amplification reaction, that of the target nucleic acid, need occur. As mentioned above, a segment of nucleic acid can be removed from the amplification product(s). Subsequently, the masses of one strand of each of the shortened amplified product and the shortened reference nucleic acid can be compared to determine the identity of a base responsible for a base composition difference, if there is any, between the shortened amplified product and the shortened reference nucleic acid.

In another embodiment, the mass of a first nucleic acid incorporating a mass-modified nucleobase is compared with the mass of a second nucleic acid. The second nucleic acid may incorporate a mass-modified nucleobase or it may not. The mass difference, if any, is compared with a matrix of possible mass differences between the two nucleic acids to determine the identity of a base responsible for a base composition difference, if any, between the two nucleic acids.

The invention will be understood further upon consideration of the following drawings, description, and claims.

### DESCRIPTION OF THE DRAWINGS

FIG. 1 is a highly schematic diagram of an amplification reaction of a wild-type target nucleic acid in the absence and in the presence of a mass-modified nucleobase.

FIG. 2 is a highly schematic diagram of an amplification reaction of a mutant target nucleic acid in the absence and in the presence of a mass-modified nucleobase.

FIG. 3 is a table of predicted mass changes for a single base change where the mass-modified nucleobase is a bromine-modified nucleobase.

FIG. 4 is a table of predicted mass changes for a single base change where the mass-modified nucleobase is a iodine-modified nucleobase.

FIG. 5 is a table showing the approximate maximum length of amplification product that can be analyzed for a single base change.

FIG. 6 is a highly schematic diagram of the design of a PCR primer containing a Type IIs restriction site.

FIG. 7 is a highly schematic diagram of the PCR product obtained with a PCR primer shown in FIG. 6.

FIG. 8 is a listing of the Bsg I digestion products shown in FIG. 7 and their calculated masses.

FIG. 9 is a representation of the mass spectrograph produced by the digestion products of FIGS. 7, 8 and 10.

FIG. 10 is a highly schematic representation of an example of Bsg I digestion products of a PCR product as shown in FIGS. 7 and 8.

FIG. 11 is a simplified representation of the mass spectrograph of FIG. 9.

FIG. 12 is a representation of the mass spectrograph produced by the digestion of an amplification product (amplification of the same target nucleic acid as in FIG. 11) which was amplified in the presence of bromine-modified nucleobases.

FIG. 13A is a representation of the mass spectrograph produced with a SNP mutation relative to the PCR product shown in FIG. 10 where the SNP mutation target is amplified separately with dTTP and with bromo-dUTP and then the amplification products are mixed together.

FIG. 13B is a highly schematic representation of an example of Bsg I digestion products for the SNP mutation PCR product described for FIG. 13A.

FIG. 14 is a highly schematic representation of a PCR primer containing a Mnl I recognition sequence.

FIG. 15 is a highly schematic representation of isolating a strand of the amplified product using asymmetric PCR.

FIG. 16 is a table of the masses from the embodiment described in FIG. 15.

FIG. 17 is a highly schematic representation of two target sequences and a ladder fragment.

FIG. 18 is a highly schematic representation of generated termination fragments.

FIG. 19 is a table of the masses of the termination fragments and the difference in mass between the fragments.

FIG. 20 is a table demonstrating mass differences between some mass-modified bases and unmodified bases.

### DETAILED DESCRIPTION OF THE INVENTION

The present invention provides methods for determining polynucleotide sequence information using a mass-modified nucleobase. As used herein, the term “nucleobase” refers to a nucleic acid monomer having a functional characteristic that allows it to be added to either a biopolymer, eg., a deoxyribonucleic acid (DNA), a ribonucleic acid (RNA), or a peptide nucleic acid (PNA), and chimeras thereof, or another nucleic acid monomer. Typically, a nucleobase includes a purine or pyrimidine base, a sugar, and one, two, or three phosphate groups. However, this term should be interpreted broadly to include any configuration of nucleic acid monomer that is capable of adding to (or actually has been added to) another monomer or a biopolymer (or is otherwise a part of a biopolymer). This term is represented herein in short-hand as a “base.” It also should be understood that “base” can refer to monomers in any form, including those previously incorporated (for example, nucleoside monophosphates) into a polymer chain even if, for example, phosphate groups are removed during incorporation. Nucleobase (or base) also can refer to compositions and concepts known to those skilled in the art.

A deoxyribose species of a nucleobase having three phosphate groups is referred to as a “dNTP;”a ribose species of a nucleobase having three phosphate groups is referred to as a “rNTP;”and a dideoxyribose species of a nucleobase having three phosphate groups is referred to as a “ddNTP,” as they are generally used by one skilled in the art. Nucleobases having specific purine or pyrimidine bases and having three phosphate groups are represented herein in short-hand as a “dATP,” “dCTP,” “dGTP,” “dTTP,” “dUTP,” “rATP,” “rCTP,” “rGTP,” “rTTP,” “rUTP,” “ddATP,” “ddCTP,” “ddGTP,” “ddTTP,” or “ddUTP.”

The term “mass-modified nucleobase” refers to a nuclebase having a mass that differs from the naturally-occurring nucleobase. Typically, a mass-modified nucleobase includes a purine or pyrimidine base, a sugar, one, two, or three phosphate groups, and a mass-modifying substituent (e.g., mass-modifier). The mass-modifying substituent can be present on, for example, the purine or pyrimidine base, the sugar, and/or the phosphodiester linkage. For example, the mass-modifying substituent can be a halogen such as a bromine or an iodine atom. The term mass-modified nucleobase should be interpreted broadly to include any configuration of nucleic acid monomer that is capable of adding to (or actually has been added to) another monomer or a biopolymer (or is otherwise a part of a biopolymer). This term is represented herein in short-hand as a “mass-modified base.”It also should be understood that “mass-modified base” can refer to mass-modified monomers in any form, including those previously incorporated (for example, mass-modified nucleoside monophosphates) into a polymer chain even if, for example, phosphate groups are removed during incorporation. Mass-modified nucleobase (or mass-modified base) also can refer to compositions and concepts known to those skilled in the art.

A deoxyribose species of mass-modified nucleobase having three phosphate groups is referred to as a “*dNTP,” and a ribose species of mass-modified nucleobase having three phosphate groups is referred to as a “*rNTP.” Mass-modified nucleobases (such as mass-modified dNTPs and rNTPs) having specific purine or pyrimidine nucleobases and having three phosphate groups are represented herein in short-hand as a “*dATP,” “*dCTP,” “*dGTP,” “*dTTP,” “*dUTP,” “*rATP,” “*rCTP,” “*rGTP,” “*rTTP,” or “*rUTP.” Mass-modified nucleobases having a specific mass-modifier, such as bromine or iodine, typically are referred to with the mass-modifier compound's name, or a version thereof, before any of the terms used above, eg., “bromo-dUTP” or “bromine mass-modified base.”

The terms “adenosine residue,” “cytidine residue,” “thymidine residue,” “guanosine residue,” and “uridine residue” are used generally to refer to the identity of a base in a sequence. For example, but without limitation, those skilled in the art use “A,” “C,” “G,” “T,” and “U” in certain circumstances to refer generally to the identity of a base in a sequence, but, for grammatical reasons, the terms “adenosine residue,” “cytidine residue,” “thymidine residue,” “guanosine residue,” and “uridine residue” can be used herein. Other terms can also be followed by “residue” to generally refer to the identity of a base in a sequence such as a bromo-uridine residue which is a bromine-modified uridine residue. These terms can be used to indicate a mass-modified base or a non-mass-modified base, depending upon the context.

I. Comparison of Nucleic Acid Incorporating a Mass-Modified Base With Nucleic Acid Not Incorporating a Mass Modified Base

In one embodiment of the invention, a first sample containing a target nucleic acid is amplified with four types of unmodified nucleobases (for example, dATP, dCTP, dGTP, and dUTP (dTTP can substitute for dUTP in some embodiments)) to produce a reference nucleic acid. Amplification is accomplished using any available amplification procedures known in the art. For example, PCR, strand displacement amplification (SDA) (see e.g. Little et al. (1999), Clinical Chemistry 45(6): 777-84, which is incorporated by reference herein), rolling circle amplification (sees e.g. Schweitzer et al. (2000), P.N.A.S. 97(18): 10113-9, which is incorporated by reference herein), nucleic acid sequence-based amplification (NASBA) (see eg., van Deursen et al. (1999), Nucleic Acids Research 27(17): 15, which is incorporated by reference herein), or reverse transcription-PCR (see e.g., Kawasaki (1990), “Amplification of RNA” in: PCR Protocols, A Guide to Methods and Applications (Innis et al., eds, Academic Press, San Diego) pp. 21-27, which is incorporated by reference herein), can be used. An amplification product can be, for example, DNA or RNA.

A second sample of the target nucleic acid is amplified in the presence of three unmodified nucleobases (for example, dATP, dCTP, and dGTP) and one mass-modified nucleobase (for example, *dUTP) to produce an amplified product. The mass-modified nucleobase can have a modification that increases the mass of the base by more than about 27 amu, preferably by about 50 amu or more. For example, a bromine-modified nucleobase, which typically involves a substitution of a bromine atom for a hydrogen atom, has a mass that is about 78.9 amu greater than the naturally-occurring base. An iodine-modified nucleobase, which typically involves a substitution of a iodine atom for a hydrogen atom, has a mass that is about 125.9 amu greater than the naturally-occurring base.

It should be noted that some amplification methods, such as PCR, produce two complementary strands of an amplification product. For the sake of clarity, unless otherwise noted, the methods described herein refer to only one of the two amplification product strands. However, the method applies equally to the second strand. Other amplification reactions, such as transcription of RNA, reverse transcription, asymmetric PCR, or extension reactions, produce only a single strand of an amplification product so this distinction is unnecessary.

Subsequent to amplification, the mass of one strand of the reference nucleic acid (amplified without mass-modified nucleobases) and the mass of one strand of the amplified product (amplified in the presence of a mass-modified nucleobase) is determined. Any of a variety of mass spectrometry techniques known in the art can be utilized to determine these masses. For example, Matrix-assisted Laser Desorption with Time of Flight (“MALDI-TOF”), electrospray ionization (“ES”), Fourier Transform (“FTIR”), or Ion Cyclotron Resonance (“ICR”) mass spectrometry can be used. See, e.g., Mass Spectrometry in Biology and Medicine(Burlingame et al., eds., Human Press, Totowa, N.J.), which is incorporated by reference herein. The mass difference, if any, between one strand of the reference nucleic acid and one strand of the amplified product is divided by the difference between the mass of the unmodified nucleobase (for example, dUTP) and the mass-modified nucleobase (for example, *dUTP) to determine the bases of a given type in the sequence which is analyzed (in this case, uridine residues). The mass difference between the unmodified nucleobase and the mass-modified nucleobase is due to the mass modification, such as the substitution of a bromine or an iodine for another atom or the addition of another mass-modifier. It should be noted that most amplification reactions, when incorporating a nucleobase having three phosphate groups, remove two phosphate groups from the nucleobase. However, the mass difference between an unmodified base having three phosphate groups and a modified base having three phosphate groups is the same as the difference between the base, less two of the three phosphate groups, and the modified base, less two of the three phosphate groups. In some situations, amplification need not occur if the sequence of a target nucleic acid is known such that mass calculations can be made based on the known sequence.

If this technique is used to compare a wild-type sequence and a mutant sequence, the procedure described above is repeated using a sample containing the mutant sequence (assuming the target nucleic acid sequence used above contains a wild-type sequence). Changes in the sequence alter the number of bases of a given type from those expected in a known normal sequence. For example, a single base change in the mutant sequence from the wild-type sequence (for example, a SNP) will change the identity of a single base between the two target nucleic acid sequences and will change the number of the mass-modified nucleobases incorporated in the mutant sequence relative to the wild-type sequence if the mass-modified base is complementary to the polymorphic base in the wild-type sequence or the mutant sequence.

Insertions, deletions, repeats, and other polymorphisms can alter the number of mass-modified bases by more than one. The number of mass-modified bases in the mutant sequence is calculated as described above and then compared to the number calculated with respect to the wild-type sequence. Any mass difference between the two sequences is due to a base composition difference of bases corresponding to the mass-modified base. From this data, the base composition difference between the initially amplified target nucleic acids is readily discernible from standard base-pairing rules. For example, if one strand of an amplified product of the mutant sequence incorporates an additional bromo-dUTP, then the mutant target sequence, which is complementary to the strand of amplified product, contains an additional adenosine residue compared to the wild-type sequence. For example, this technique can be used to detect a mutant nucleic acid sequence in an unknown sample by comparison with a wild-type sequence in a known sample, or vice versa. Additionally, the number of mass-modified bases incorporated into the amplification product that are attributable to the primer (ie., the sequence complementary to the primer) can be easily accounted for in calculations because the sequence of a primer typically is known.

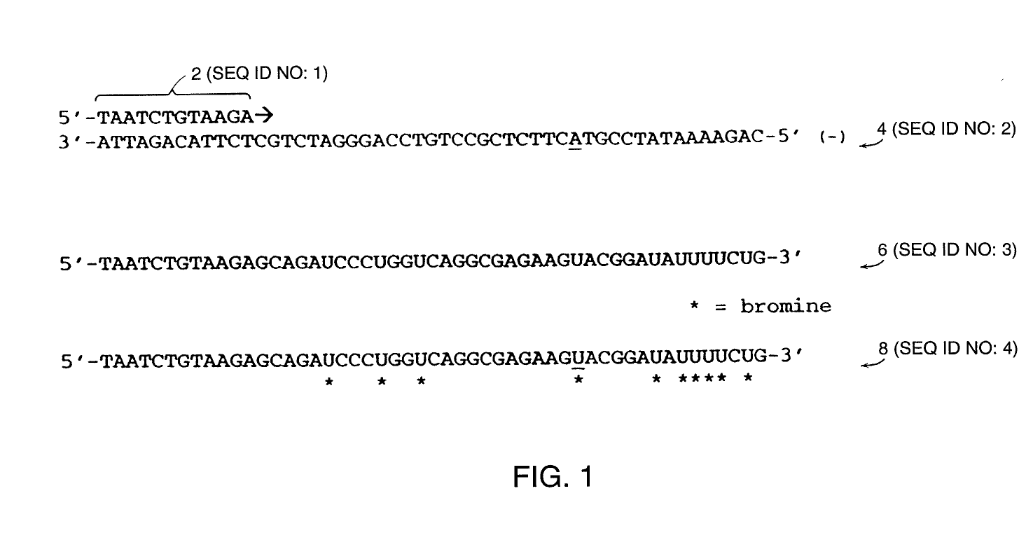

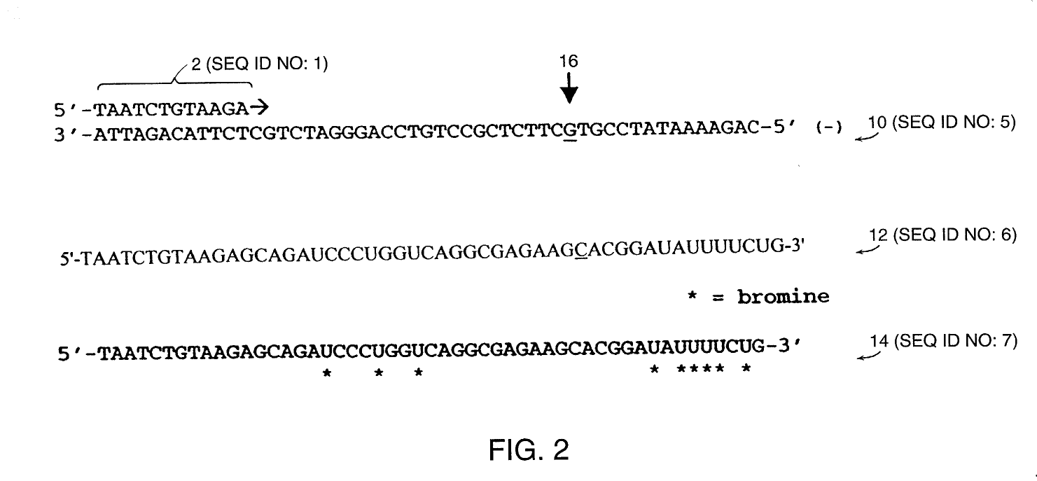

One example of this embodiment is shown in FIGS. 1 and 2. An amplification product is created using standard PCR methods and reagents. Although the amplification produces two complementary strands of a PCR product, the method is described here with respect to only one of the two product strands for the sake of clarity. The method applies equally to the second strand. The amplification reactions are performed in pairs. The amplification reaction is performed first with four dNTPs: dATP, dCTP, dGTP, and dUTP. For example, a forward primer 2 (SEQ ID NO: 1) is used to amplify a target strand 4 (SEQ ID NO: 2) (the reverse primer is not shown). One strand of the resulting amplification product 6 (SEQ ID NO: 3) (i.e., one strand of the reference nucleic acid) incorporates these dNTPs as dictated by the principles of Watson-Crick base pairing and amplification reactions. Then, a second amplification reaction is performed. This amplification reaction substitutes one of the dNTPs with a mass-modified nucleobase, for example substituting bromo-dUTP for dUTP. One strand of the resulting second amplified product 8 (SEQ ID NO: 4) (i.e., one strand of the amplified product) incorporates the three dNTPs and the one *dNTP, i.e., *dUTP.

In the example shown in FIG. 1, the wild-type sequence (i e., the sequence following the PCR primer 2 in the amplification product 6) contains ten uridine residues. The number of uridine residues is determined by measuring the mass of the strand of the amplification product 8 incorporating the mass-modified bases and the mass of the strand of the amplification product 6 which does not incorporate mass-modified bases. The mass difference between the strand of the amplification product 8 incorporating the mass-modified bases and the strand of the amplification product 6 which does not incorporate mass-modified bases, in this case about 790 amu, is determined using mass spectrometry. Subsequently, this mass difference is divided by the mass difference between the unmodified base (dUTP) and the mass-modified base (*dUTP), in this case about +78.9 amu for a bromine mass-modified base, to provide the number of corresponding base residues in the wild type sequence. The mass difference between the two strands of the amplification products 6, 8 is due to the bromines on the ten bromo-dUTPs incorporated into the one strand of the amplified product 8. This procedure yields the expected total of uridine residues, namely ten. The number of other bases can be determined by repeating the same process, but using a different mass-modified base (e., a mass-modified dCTP).

The number of mass-modified bases (and hence, the number of bases of that type which can be used to determine the number of bases present in the original target sequence through base pairing rules) can be compared with mass data from another sample containing a mutant sequence. As shown in FIG. 2, a mutant target sequence 10 (SEQ ID NO: 5) contains a guanosine residue rather than an adenosine residue at the polymorphic target site 16 (underlined). Utilizing the procedure outlined above, the mutant target sequence 10 is amplified with the forward primer 2 (again, the reverse primer is not shown) in the absence of a mass-modified base and in the presence of a mass-modified base (bromo-dUTP). The amplification reactions produce an amplification product which does not incorporate the mass-modified base (i.e., the reference nucleic acid) and an amplification product that does incorporate a mass-modified base (ie., the amplified product). Again, one strand of the reference nucleic acid 12 (SEQ ID NO: 6) (which does not incorporate the mass-modified base) and one strand of the amplified product 14 (SEQ ID NO: 7) (which does incorporate a mass-modified base) are shown. Because the mutant target sequence 10 contains a guanosine residue rather than an adenosine residue, the strand of the amplified product 14, which is complementary to the mutant target sequence 10, will incorporate one less mass-modified dUTP. That is, only nine mass-modified dUTPs are incorporated, compared to ten for the wild-type. In fact, the mass difference between the two strands of the two amplification products 12, 14 is about 711 amu. This mass difference is divided by the mass difference between the unmodified base (dUTP) and the mass-modified base (*dUTP), in this case about 78.9 amu, to obtain the number of mass-modified dUTPs incorporated into the strand of the amplification product, namely, nine. Note that the mass increase due to bromine incorporation is about 79 amu less than that obtained with the wild-type sequence. These results reveal the loss of one uridine residue in the strand of the amplified product 14. Because the amplified product 14 is complementary to the original target sequence, base pairing rules dictate that the adenosine residue at the polymorphic site in the wild-type sequence changed to another base in the mutant sequence which is not complementary to a uridine residue.

It should be noted that the identity of a changed base can be discerned using this method. For example, if the mass-modified base is a bromo-dUTP and the difference in mass between an amplification product of a wild type nucleic acid incorporating the mass-modified base and an amplification reaction of a mutant nucleic acid incorporating the mass-modified base is about 79 amu, the mutant amplification product incorporated one more bromo-UTP and the original mutant nucleic acid had one more adenosine residue than the original wild type nucleic acid. If the experiment is then run with unmodified and bromo-modified dCTPs and it is found that one more mass-modified base was incorporated into the wild type amplification product than the mutant amplification product, it can be surmized that the wild type nucleic acid contained a guanosine residue rather than the adenosine residue.

II. Comparison of Two Nucleic Acids Incorporating a Mass-Modified Base

The method described above typically utilizes four amplification reactions (PCR with and without the mass-modified base for both the wild-type and mutant target sequences) to disclose the mutation. However, two amplification reactions may be sufficient to reveal a mutation. Specifically, a mutation can be detected by comparing the masses of the amplification products resulting from amplifying two nucleic acid sequences (for example, mutant and wild type target sequences) with the same mass-modified base. This method does not directly reveal the exact number of bases in each sequence, but discloses the net difference between the two target nucleic acid sequences. Thus, in one embodiment of this method, a first sample containing a target nucleic acid is amplified in the presence of three unmodified nucleobases (for example, dATP, dCTP, and dGTP) and one mass-modified nucleobase (for example, *dUTP) and a second nucleic acid sample is amplified in the presence of three unmodified nucleobases (for example, dATP, dCTP, and dGTP) and one mass-modified nucleobase (for example, *dUTP).

As described above, amplification can occur by any number of amplification reactions. Many amplification methods, such as PCR, produce two complementary strands of an amplification product. For the sake of clarity, unless otherwise noted, the methods described herein refer to only one of the two amplification product strands. However, the method applies equally to the second strand. Other amplification reactions, such as transcription of RNA, reverse transcription, asymmetric PCR, or an extension reaction produce only a single strand of an amplification product so this distinction is unnecessary. An amplification product can be, for example, DNA or RNA. The masses of one strand of each of these amplification products is obtained using mass spectrometry. As described above, many different mass-spectrometry techniques can be used.

The mass difference, if any, between one strand of the first amplification product (for example, one strand of the amplified product) and one strand of the second amplification product (for example, one strand of the reference nucleic acid) is compared to a matrix of expected mass differences between the strands of amplification products when particular base changes occur in order to determine whether the two amplification products have a different base composition for a base of a given type (in this case, uridine residues). The second amplification step need not occur, as the known or calculated mass of one strand of a reference nucleic acid (of known sequence or base composition and which incorporates mass-modified bases) can be used or one or both strands of the reference nucleic acid incorporating the mass-modified base can be provided with, for example, a kit. Accordingly, only one amplification reaction, that of a target nucleic acid, need occur.

A comparison of one strand of each of the amplification products amplified in the presence of the mass-modified base 8, 14, as shown in FIGS. 1 and 2, respectively, illustrates this embodiment. There is a difference of about 80 amu between these strands. Part of the about 80 amu difference (about 79 amu) is due to having one fewer bromo-dUTP incorporated in the one strand of amplified product 14 complementary to the mutant target sequence than in the one strand of the amplified product 8 from the wild-type target sequence. This difference corresponds to a loss of a bromine due to having one fewer incorporated bromo-dUTP. The other part of the about 80 amu difference is obtained because there was an additional mass loss of about 1 amu because the uridine residue changed to a cytidine residue in the PCR product 14 (i.e., the mass difference between an unmodified uridine residue and an unmodified cytidine residue is about 1 amu). A net loss of about 80 amu from one strand of the amplification product 8 of the wild-type target sequence uniquely indicates a uridine residue to cytidine residue base substitution in the one strand of the amplified product 14 of the mutant target sequence. This information is then used to determine the base change in the original target sequence through base-pairing rules. Here, it can be determined that an adenosine residue in the wild-type target sequence (complementary to the bromo-dUTP) changed to a guanosine residue in the mutant target sequence (complementary to the dCTP).

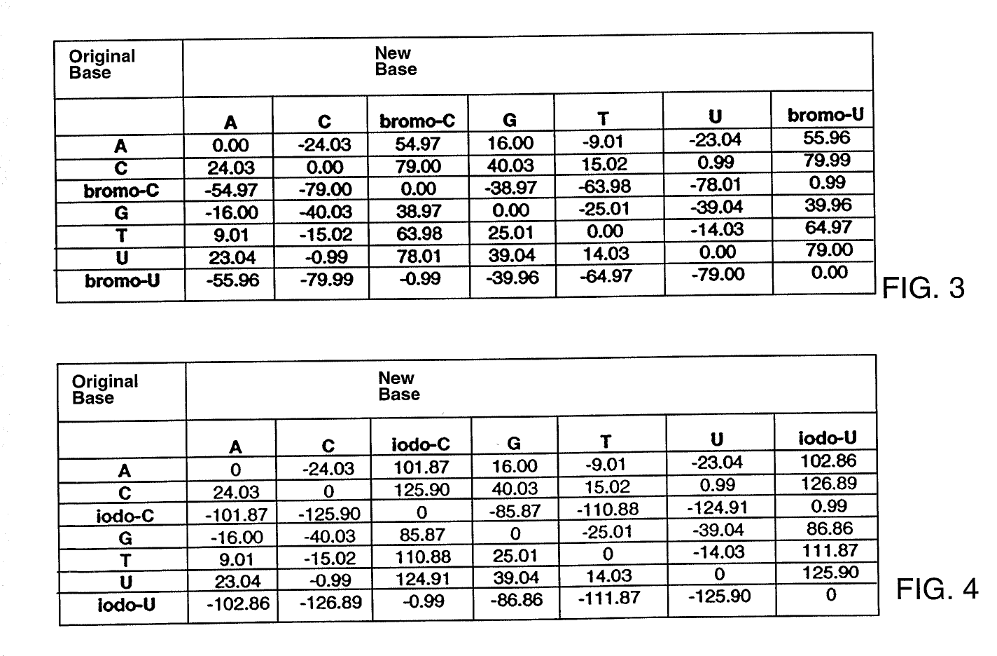

Other base changes produce different net gains or losses. These gains or losses are set forth in FIG. 3 for bromine-modified mass-modified bases and in FIG. 4 for iodine-modified mass-modified bases. The matrices shown in FIGS. 3 and 4 list the “original base” found in the amplification product from the “original” nucleic acid (e, an amplified wild-type target sequence) down one side of the matrix and list the “new” base found in the “new” nucleic acid (eg,an amplified mutant target sequence). At the intersection of each row and column, the mass difference caused by the original base changing to the new base is listed. For example, in FIG. 3, the intersection of “bromo-U” as an original base with “C” as a new base shows a mass, difference between the two amplification products of about−80 amu (−79.99 amu in FIG. 3), which agrees with the calculation described above. The other cells in this matrix also describe the loss or gain (indicated by a “−” or a “+”, respectively, preceding the numeral) of mass when one base changes to another base. FIGS. 3 and 4 set forth a matrix of mass differences when there is a single base change between two nucleic acids. The same principle holds true when there are two or more base changes between two nucleic acids. The matrix is more complicated due to the increased number of possible permutations but is calculable based on the principle described above.

If a mass change is measured with sufficient mass accuracy, both the original base and new base can be determined using FIGS. 3 and 4, for example, as a look-up table. Alternatively, such matrices can be embodied in a computer application which can automatically make calculations, particularly in the cases where more than one base change is involved. For example, using bromo-dUTP as the mass-modified base incorporated into an amplified nucleic acid, an adenosine residue (found in a first amplified nucleic acid) to a bromo-uridine residue (found in a second amplified nucleic acid) change results in a mass increase of about 55.96 amu, while a guanosine residue (found in a first amplified nucleic acid) to a bromo-cytidine residue (found in a second amplified nucleic acid) change results in a mass increase of about 38.97 amu. In order to determine both alleles, this method requires relatively high mass accuracy.

III. Additional Features of the Invention

A. Resolution Considerations

Mass-modified dCTPs and dUTPs which are suitable for use in methods of the invention are currently commercially available. With only these suitable mass-modified deoxyribose base types identified, it is typically necessary to type both strands of the amplified product to discover all possible mutations. However, it should be appreciated that, theoretically, any base (for example, any deoxyribose, ribose, or dideoxyribose nucleobase) can be mass-modified to produce suitable mass-modified bases in accordance with the invention, as more fully described below. Nevertheless, most of the protocols discussed below type both strands of the PCR product in a single step. However, if, for example, suitable mass-modified dATPs, dGTPs, and/or dTTPs are identified, it may not be necessary to type both strands of an amplified product. Without suitable mass-modified dGTP and mass-modified dATP, it is not possible to directly analyze one strand of nucleic acid for mutations in all four bases. However, analysis of cytidine residues or uridine residues in a complementary strand discloses guanosine residues or adenosine residues in the original strand. Additionally, base additions, deletions, or changes in the number of base repeats (for example, -CA- repeats) in PCR products can be identified through the first base-counting protocol described above that typically uses four amplification reactions.

For mutation discovery, it is often desirable to examine relatively long sequences to find the maximum number of mutations in the minimum number of assays. Thus, it is useful to know the approximate maximum analyzable length of an amplification product using techniques according to the invention. The length limit of a base-counting assay can be estimated on the basis of resolving power of the instrument. Of all the possible mutations, single base changes (especially where one base changes to another base that is not complementary to the mass-modified base being used) cause the smallest mass shift. The heavier the mass-modifier, and the higher the instrument resolution, the longer the length of amplification product that can be examined. The DE-Voyager™ Workstation (PE Biosystems, Foster City, Calif.), a mass spectrometer system, typically produces resolution (m/Δm, where m refers to the mass of a peak and Δm refers to the width of the peak at one-half the height of the peak) of approximately 700 for PCR products. For example, if the mass of one peak is 70,000 amu and the width of the peak at one-half the height of the peak is 100 amu, then m/Δm is 700.

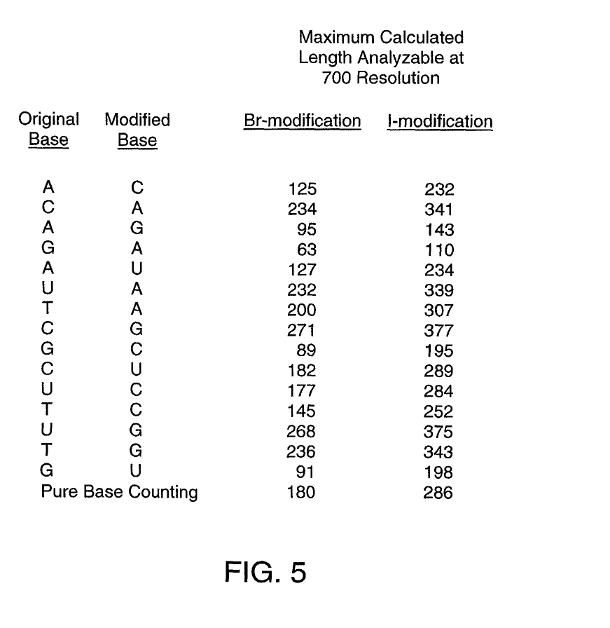

If amplified mutant and wild-type targets are compared, both amplified in the presence of a mass-modified base, the maximum length of product that can be analyzed will depend upon the allelic pair being analyzed, because some mutations will cause a greater mass shift than others. For example, when analyzing a heterozygote, each peak (representing the wild type or mutant allele) has to be resolved enough to measure each one. However, lower resolution is acceptable for homozygotes, as there will be one peak to measure. Also, thymidine residues and uridine residues represent the same allele but have different masses. Under these conditions, the calculated maximum length of an amplification product that can be analyzed for a single base change varies from about 63 to about 271 bases with a bromine mass-modified base (assuming an average mass of 308 amu per base) and about 110 to about 377 bases with an iodine mass-modified base. These maximum lengths are shown in FIG. 5. Generally, these lengths are calculated according to a mathematical formula where maximum analyzable length equals resolution multiplied by the difference in mass between two strands with that product divided by the average mass of a mononucleotide. For example, in the first row, an A to bromo-C mutation causes about a +54.97 amu (see FIG. 3) mass change, and the maximum analyzable length of an amplification product is calculated to be about 125 bases (125 bases=700 m/Δm×(54.97 amu÷308 amu/base)). If the technique employing four separate amplification reactions is conducted (“Pure Base Counting” in FIG. 5), all single base changes cause the same mass shift with the same mass-modified base. In this case, the maximum calculated length of PCR product that could be analyzed by pure base counting at a resolution of 700 is about 180 bases with a bromine mass-modified base and about 286 bases with iodine mass-modified base. In practice, limitations of sensitivity with current MALDI-TOF instrumentation, rather than resolution, may create an upper limit to the length of analyzable sequence of about less than 100 bases.

B. Purines and Pyrimidines

Sites in DNA are represented by pyrimidine-purine base pairs, so that a mutation at a site will alter the pyrimidine count in one of the two strands of the target DNA. Methods according to the invention are intended to detect all possible base combinations. At one location on one strand, there are six possible biallelic base combinations (for example, SNP's), five of which include one pyrimidine in the pair (A/C, A/T, C/G, C/T, G/T) and only one that does not (A/G). These six pairs result in 12 possible mass changes, depending on the direction of the base mutations (for example, an A to T change is regarded as a separate type from a T to A change). The five combinations which include a pyrimidine can all be detected by analysis of one strand of the amplified product. Thus, at any one location on any one strand, five of the six possible mutations can be detected by incorporation of a mass-modified pyrimidine (i.e., either the wild type or the mutant is a pyrimidine). If only mass-modified pyrimidines are available, the A/G combination can be analyzed indirectly through analysis of the T to C mutation in the complementary strand.

Although it should be uncommon, except in highly polymorphic regions, it should be understood that it is possible to have two mutations that effectively cancel each other. For example, separate A to G and G to A mutations within the same sequence will not alter the net base count. If many different mutations occur, many will nearly cancel each other out and so the net mass change may not be detectable. Because SNP's occur at a frequency of about 1 in about 500 bases, the chance of a second SNP occurring within 50 bases of a first SNP is about 10%. However, the chance that the second SNP would cancel out the first SNP is lower than 1 in 10 because such chance also depends on the frequency of the mutation of the second SNP (typically between about 1% and about 50%) and whether the nature of the mutation would increase or decrease the measured mass difference (typically, about 50% of the time, it would not negate the first SNP, but would add to it). The chances of off-setting second SNP's can be reduced by reducing the length of the product examined, at the expense of having to run more samples to cover longer total sequence lengths. The extreme case is using the technique to characterize a sample DNA length of only a single base, where offsetting errors disappear. Occasional errors can be tolerated in screening techniques, but errors should be minimized when performing molecular diagnostics when diagnosing a patient.

C. Selection Criteria for Mass-Modified Bases

There are several criteria that can be used (either alone or in any combination) for choosing and/or synthesizing a mass-modified base for use in methods according to the invention. For example, the mass-modified base should be efficiently incorporated during an amplification reaction, substituting as close to 100% as possible for the unmodified base in the amplification reaction (i e., the mass-modified base effectively acts as an unmodified base during an amplification reaction). One hundred percent substitution is preferred. One way to define 100% substitution and to test a compound to determine if it is suitable as a mass-modified base is as to run PCR with a mass-modified base in the absence of the corresponding unmodified base. If amplification with the mass-modified base occurs and produces a detectable amount of amplification product, then the mass modified base is incorporated into the product about as well as the unmodified base (i.e., substitutes at about 100%) and can be useful in methods and kits according to the invention. If it meets this criteria, it typically means that the mass-modified base is recognized by the polymerase and that the mass-modified base does not interfere with amplification product, which incorporates the mass-modified base, from serving efficiently as a template for subsequent rounds of PCR.

It would be considered acceptable to provide a higher concentration of the mass-modified base in one reaction than is provided for the unmodified base in the comparative reaction, if necessary to obtain adequate PCR yields (i.e., to “push” the reaction forward so that the mass-modified base substitutes for the unmodified base as close to 100% as possible when the two reactions are compared). This situation might occur if the polymerase had a weaker affinity for the mass-modified base as compared with the unmodified base. Raising the concentration of the mass-modified base relative to that used for an unmodified base in the comparative reaction would increase the reaction rate so that the mass-modified base would substitute for the unmodified base at a similar incorporation rate.

Also, base-pairing rules should be obeyed such that the mass-modified base only should be incorporated at sites directed by its proper complement. Bromo-dUTP, for example, should only be incorporated opposite an adenosine residue in the target sequence. The error rate can be determined by sequencing cloned DNA. Additionally, the mass-modified base should be stable and not chemically degrade under the amplification conditions, including high temperatures such as those used in PCR. The mass-modified base should not interfere with any subsequent steps in practice of the methods according to the invention and should induce a desirable mass shift from the unmodified base (more than about 27 amu, and preferably more than about 50 amu; in many instances, the mass shift can be about 50 to about 300 amu or more). Further, the mass-modified base should not induce excessive mass heterogeneity and should not reduce signal response.

Some mass-modifiers do not have an exact mass, but are a mixture of masses. For example, bromine does not have an exact mass of 80 amu, but is about a 50:50 mixture of the two major naturally-occurring isotopes of bromine with atomic weights of about 79 amu and about 81 amu. When multiple mass-modified bases are incorporated into an amplification product, there is a statistical mass broadening because, by chance, some molecules have more or less of the heavy isotope. With bromine, the two isotopes do not add much broadening (i.e., mass heterogeneity) because the two isotopes are 2 amu apart, but iodine is better than bromine in this respect because iodine is almost entirely a single isotope. When the mass-modifier has more than one atom (for example, organic carbon chains), the broadening can be greatly increased because each of the atoms might include isotopic variants. Additionally, some chemical groups interfere with desorption and/or ionization in a mass spectrometry device. For example, polar groups like phosphates generally ionize more poorly in MALDI-TOF and give less signal (i.e., reduces signal response). Such interference varies with each mass spectrometry technique.

These principles can be applied to other amplification reactions to compare amplification incorporating a proposed mass-modified base and amplification incorporating the corresponding unmodified base. Additionally, similar rules apply to RNA synthesis, except that the RNA polymerase is continually reading from a DNA strand, so the guideline about the mass-modified base not interfering with serving as a target does not apply.

Many commercially available mass-modified bases (whether the bases are sold with, or are subsequently attached to, modifying groups, such as fluorescent dyes and haptens), will not substitute 100% for unmodified bases in amplification reactions such as PCR. One reason these mass-modified bases will not substitute 100% for unmodified bases may be that they are not efficiently incorporated by DNA polymerases. Another reason may be that even if incorporated into the amplification product, such mass-modified bases interfere with further amplification, for example, because such mass-modified bases, once incorporated into an amplification product, are not satisfactory templates for further replication. For example, bulky fluorescent labels such as fluorescein may interfere. Typically, such mass-modified bases can be incorporated by PCR only if mixed with a vast excess of the unmodified base, creating a situation where a relatively low proportion of the incorporated bases are mass-modified bases. Possibly, many dye and hapten mass-modifiers either do not fit into the active site of a DNA polymerase or, once in the template, interfere with the hydrogen bonding required to form base pairs.

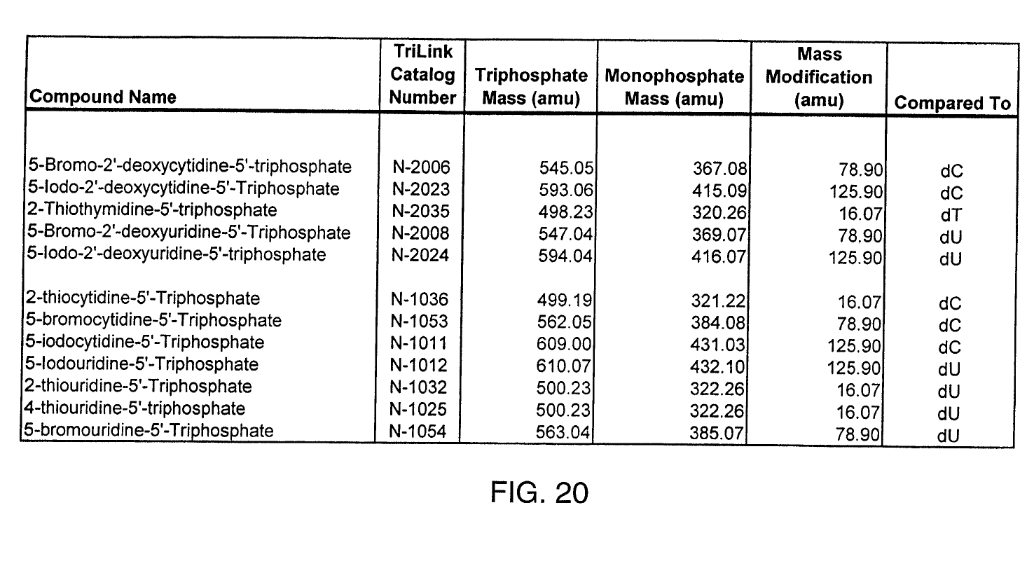

Bromo-dUTP or iodo-dUTP can substitute well for dUTP in PCR. A number of bromine and iodine mass-modified dNTPs are commercially available and were screened for their ability to support PCR. The following mass-modified dNTPs were found to support a model PCR employing AmpliTaq® DNA polymerase (a TAQ DNA polymerase available from Applied Biosystems, Foster City, Calif.) with about 100% substitution for the corresponding unmodified dNTP. Incorporation was equally efficient with AmpliTaq® DNA polymerase and Tth DNA polymerase (Applied Biosystems, Foster City, Calif.). The tested mass-modified bases were obtained from TriLink BioTechnologies, Inc., San Diego, Calif. These mass-modified bases are 5-Bromo -2′-deoxycytidine-5′-triphosphate (Trilink N-2006); 5-Iodo-2′-deoxycytidine-5′-Triphosphate (Trilink N-2023); 5-Iodo-2′-deoxyuridine-5′-triphosphate (Trilink N-2024); 5-Bromo -2′-deoxyuridine-5′-Triphosphate (Trilink N-2008); and 2-Thiothymidine-5′-triphosphate (Trilink N-2035). Additionally, ribonucleoside mass-modified bases can be used in those reactions using rNTPs. For example, 5-iodocytidine-5′-Triphosphate (Trilink N-1011); 5-Iodouridine -5′-Triphosphate (Trilink N-1012); 2-thiouridine-5′-Triphosphate (Trilink N-1032); 4-thiouridine-5′-triphosphate (Trilink N-1025); 2-thiocytidine-5′-Triphosphate (Trilink N-1036); 5-bromocytidine-5′-Triphosphate (Trilink N-1053); and 5-bromouridine-5′-Triphosphate (Trilink N-1054) incorporate efficiently using T7 and T3 RNA polymerase. Other halogen-modified bases may be useful as well. FIG. 20 is a table containing the names of certain mass-modified bases, the atomic weight of the mass-modified bases in a triphosphate form, the atomic weight of the mass-modified bases in a monophosphate form, and the mass difference between each of the mass-modified bases in the triphosphate form and its corresponding unmodified base in the triphosphate form. This mass difference is the same as the mass difference between each of the mass-modified bases in the monophosphate form and its corresponding unmodified base in the monophosphate form.

While bromine and iodine have appropriate masses for use as mass modifiers, their atomic radii are relatively small. Accordingly, their substitution for hydrogen in a mass-modified base may interfere less with amplification reactions than some other mass modifiers with larger atomic radii and/or size (for example hydrocarbon chains). This physical structure might be one factor to consider when searching for other mass-modifiers. Bromine substitutions add about 78.9 amu for each one incorporated into a base relative to the same base without bromine. Iodine substitutions add about 125.9 amu for each one incorporated into a base relative to the same base without iodine. These added masses are in a useful range of masses. Thiol-substituted dTTP also was useful, but sulfur added only about 16 amu (sulfur is about 32 amu and was substituted for an oxygen, which is about 16 amu, in the mass-modified base). Sixteen amu is, for most applications, a smaller mass increase than is desirable. Bromine substitutions cause some isotopic broadening because the natural isotopic composition of bromine is about 50% bromine-79 and about 50% bromine-81. However, bromines do not substantially interfere with MALDI mass-spectrometry measurements. Iodine is nearly 100% iodine-127 (about 126.9 amu). Iodine also does not substantially interfere with MALDI mass-spectrometry measurements.

Bases modified with stable isotopes (for example, the elemental constituents of the base are replaced with isotopic variants) can be incorporated during an amplification reaction fairly efficiently relative to incorporation of non-modified bases. However, such modifications add relatively low mass per nucleotide (9 amu to 27 amu added to an amplified product), which can be difficult to resolve or measure accurately, and the modified compounds are very difficult and extremely expensive to prepare. The highest mass shifts are obtained if the products are labeled with deuterium (with complete substitution of deuterium for every hydrogen), in addition to carbon-13 and nitrogen-15, but in this case, some of the hydrogens are exchangeable and the PCR must be carried out in deuterium-substituted solvents to avoid loss of the deuterium label. Also, PCR products can be generated with 7-deazapurine residues, however, deaza-labeled bases are not satisfactory as mass labels since they differ by only 1 amu from the mass of the corresponding unmodified bases.

While halogen-modified purines (adenosine and guanine residues) can be incorporated into DNA in vivo, in vitro incorporation of them in PCR products with thermophillic DNA polymerases was inefficient. A mass-modified purine, 8-chloro-2′-deoxyadenosine-5′-triphosphate also was tested as a substitute for dATP. It supported primer extensions in a model system, but with some premature terminations. It did not support PCR. Possibly, it is more difficult to incorporate mass-modified purine bases than it is to incorporate mass-modified pyrimidine bases with thermophillic DNA polymerases. For example, the position of the mass-modification might interfere with hydrogen bonding to a pyrimidine.

D. Removal of Nucleic Acid Segments

The methods outlined above can be combined with additional steps. For example, PCR primers contribute mass to the PCR product, but in the PCR product there is little useful sequence information in the primers or in the sequence complementary to the primers. Removing these sequences results in a shortened amplification product (in contrast to the full-length amplification product) that can be examined with higher resolution, greater mass accuracy, and greater sensitivity than the corresponding full-length amplification products. To the extent that a shortened amplification product is examined, any of the methods described above are applicable.

After a segment is removed from an amplified product and the same segment is removed from a reference nucleic acid, the masses of one or more strands of the shortened amplified product and one or more strands of the shortened reference nucleic acid are compared. If the shortened amplified product incorporates mass-modified bases and the shortened reference nucleic acid does not, then the number of mass-modified bases incorporated into one or more strands of the shortened amplified product can be determined as described above for the full-length amplified product. If both the shortened amplified product and the shortened reference nucleic acid incorporate mass-modified bases, then the identity of the base responsible for a base composition difference, if any, between the one or more strands of the two shortened products can be determined as described above for the full-length amplified product. It also is possible to subtract the mass of the removed segment from the mass of the full-length reference nucleic acid in instances where a second amplification reaction in not conducted.

One way to remove primer sequences is to use PCR primers that contain a 5′ sequence which is a recognition site for a type IIs restriction endonuclease. This type of restriction endonuclease cleaves several bases downstream from its recognition site. For example, Bsg I restriction endonuclease (Catalog#R0559, available from New England BioLabs, Beverley, Mass.) cleaves double stranded nucleic acids fourteen and sixteen bases downstream from its recognition site (a staggered cut). Thus, if a core primer sequence (typically complementary to a target nucleic acid), which follows the 5′ embedded restriction enzyme recognition sequence, is sixteen bases long, then the entire primer will be excised from the PCR product, as well as most of its complement (minus two bases due to the staggered cutting).

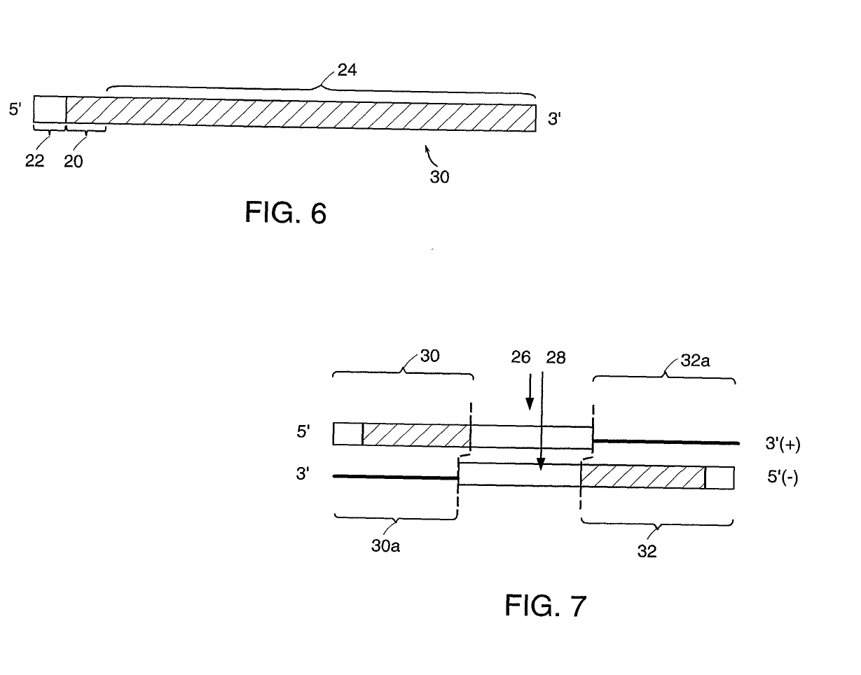

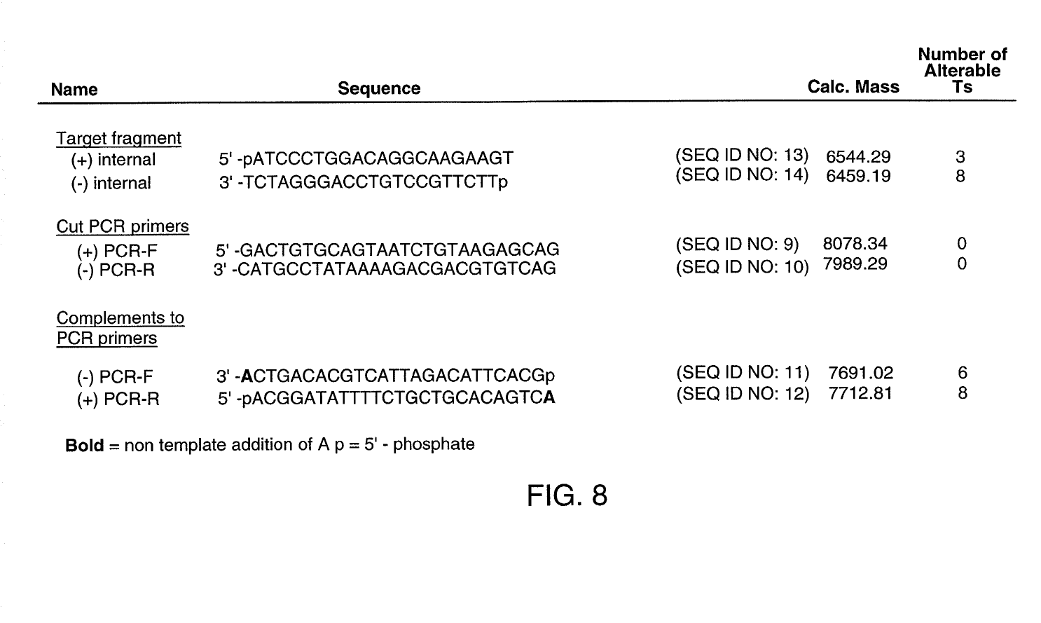

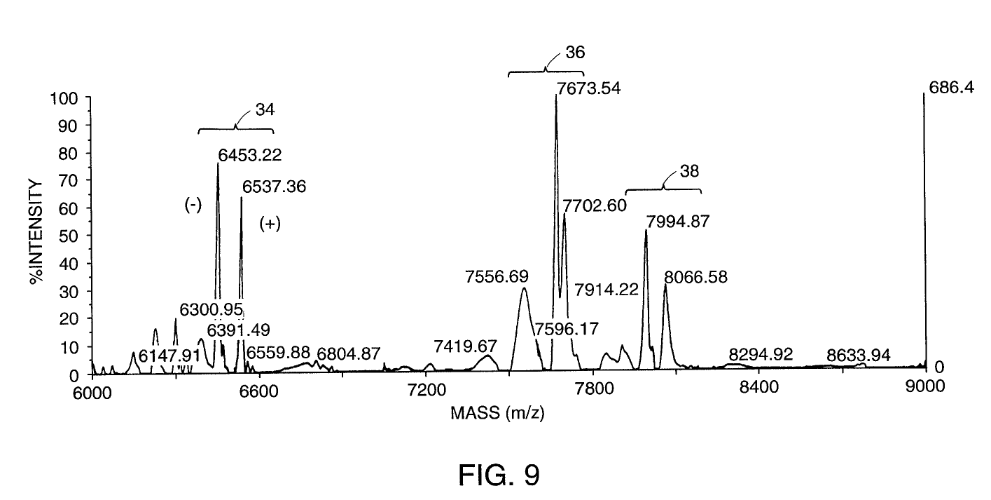

This technique is illustrated in FIGS. 6, 7, and 8. A 71-base pair (“bp”) target sequence was amplified with forward and reverse primers 30, 32 as shown in FIG. 7. Using the primer 30 in FIG. 6 as an example, the primer contains a core sequence 24 that is sixteen bases long. Located at the 5′ end of the core sequence 24 is a six base recognition sequence 20 (in this case it is GTGCAG (SEQ ID NO: 8)) and 5′ to the recognition sequence 20 is a four base cap sequence 22 (which can be any sequence). A cap sequence, it is thought, allows the enzyme to sit better on the nucleic acid as opposed to having the recognition sequence at the extreme 5′ end of the primer. The length and sequence of the cap sequence can be empirically determined for any enzyme that is used. Thus, the primer 30 is designed such that the entire primer sequence is removed with Bsg I digestion (which is typically done prior to mass spectrometry analysis). The fragments generated with Bsg I digestion are shown in FIG. 7 and listed in FIG. 8. Cuts in the amplification product are shown by dashed lines. The primers 30, 32 (SEQ ID NOS: 9 and 10, respectively) are excised (“Cut PCR primers” in FIG. 8) as well as most of their complements 30a, 32a (SEQ ID NOS: 11 and 12, respectively) (“Complements to the PCR primers” in FIG. 8, each of which is two bases shorter than the excised primer due to staggered cuttings). Two strands of the shortened amplification product 26, 28 (SEQ ID NOS: 13 and 14 , respectively) (“Target fragments” in FIG. 8) remain. One or both strands of the shortened amplification product can be isolated before mass spectrometry analysis or, as shown in FIG. 9, all of the digestion products can be analyzed with mass spectrometry at the same time. FIG. 8 lists the expected masses of the various fragments (“Calc. Mass”) and the number of thymidine residues in each fragment which can be replaced using a bromo-dUTP when amplification takes place in the presence of the mass-modified base instead of dTTP (“Number of Alterable Ts”).

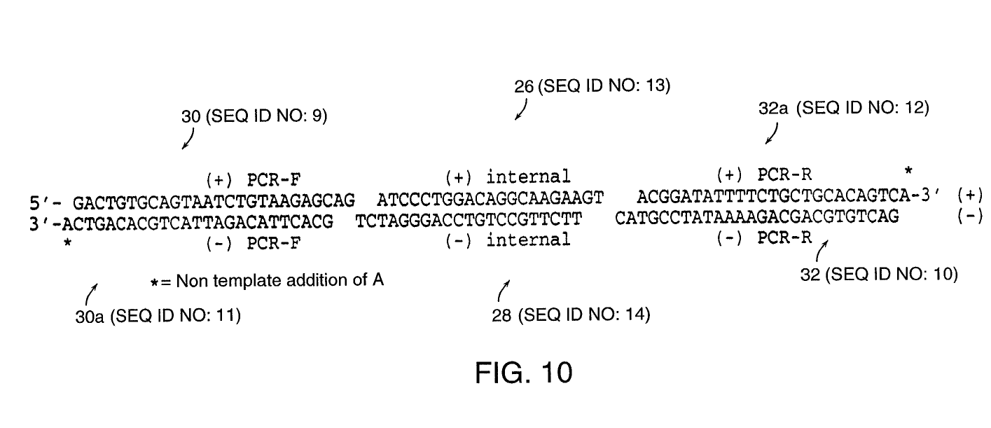

As described above, PCR product was generated using dATP, dCTP, dGTP, and dTTP, and the double-stranded PCR product was digested with Bsg I, producing six primary fragments (FIGS. 8 and 10). The two fragments lowest in mass are the positive and negative strand of the sequences of interest 26, 28 (located between the PCR primers). The measured masses of this pair of strands represented as a pair of peaks 34 in FIG. 9 (6537.36 amu for the positive strand and 6453.22 amu for the negative strand) match the expected masses (6544.29 amu, 6459.19 amu, as shown in FIG. 8). The two highest mass fragments are the cleaved PCR primer sequences from the positive 30 and negative 32 strands, labeled (+) PCR-F and (−) PCR-R, respectively. The measured masses of these strands, represented as a pair of peaks 38 in FIG. 9 (8066.58 amu for (+) PCR-F and 7994.87 amu for (−) PCR-R), match the expected masses (8078.34 amu, 7989.29 amu, as shown in FIG. 8). In this example, unincorporated PCR primers and the primer sequence excised by digestion happen to be identical, but need not be so. If they were not identical, additional peaks would be visible on a mass spectrograph and steps could be taken to remove unwanted fragments to reduce the number of peaks. The middle pair of peaks 36 corresponds to the sequences that are complementary to the PCR primers 30a, 32a (but which are two bases shorter than the primers due to staggered cutting) and their measured masses (7673.54 amu for (−) PCR-F and 7702.60 amu for (+) PCR-R) match well with the expected masses shown in FIG. 8 (7691.02 amu, 7712.81 amu), but provide uninformative sequence information.

In this example, when the PCR is carried out with bromo-dUTP, all of the fragments of the amplification product, except for the PCR primers that are incorporated into the amplification product, will contain bromine mass-modified residues because bromo-dUTP is incorporated into the product extended from the 3′ end of the primer, but not into the sequence occupied by the PCR primer itself. (Note that in FIG. 8 the “Number of Alterable Ts” column indicates that (+) PCR-F and (−) PCR-R have no alterable thymidine residues because bromo-dUTP is not incorporated into the primers). The number of bromine mass-modified bases incorporated in the internal fragments is determined by the target sequence to be amplified, and the number of bromine mass-modified bases incorporated in the fragments complementary to the primer are determined and fixed by the primer sequence. In this example, the PCR primers are removed and cannot incorporate any bromine mass-modified bases, and, if the primer were made shorter on the 3′ end, some of the internal sequence would end up in the primer fragment.

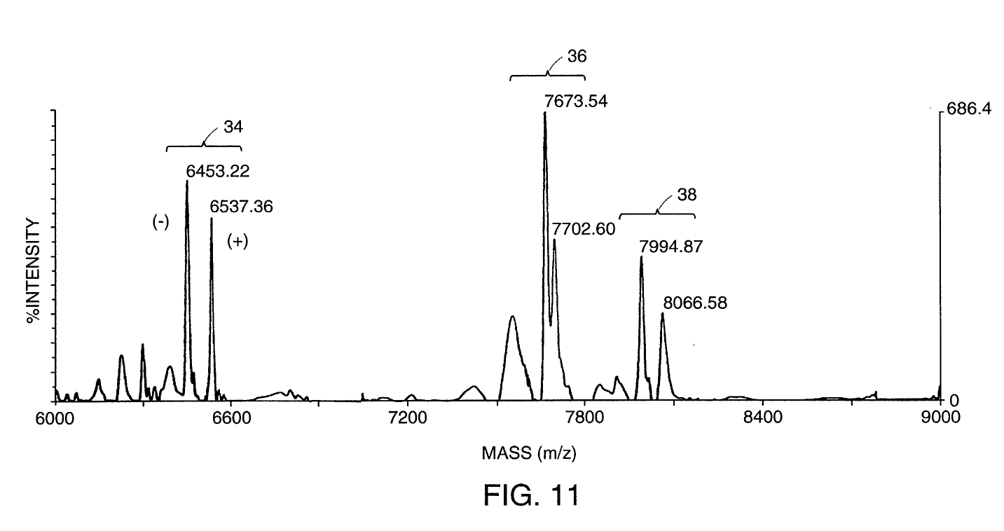

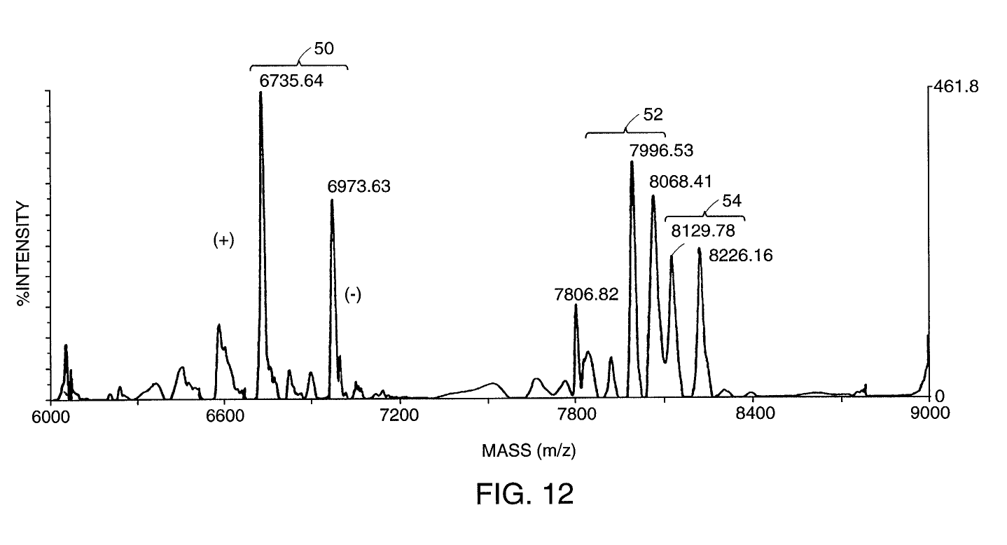

When the same amplification reaction and enzyme digestion was performed in the presence of bromo-dUTP, rather than dTTP, as expected, all fragments except the excised PCR primers became heavier. The mass spectrographic peaks for the fragments of the amplification product incorporating mass-modified bases is shown in FIG. 12. FIG. 11 is a slightly simplified version of FIG. 9 and is provided for comparison to FIG. 12. The peak pairs in FIG. 12 for the internal fragments 50, the PCR primer complements 52, and the PCR primers 54 are for the same fragment peak pairs 34, 36, 38, respectively, as shown in FIGS. 11 and 9.

The mass increase of the (+) strand internal fragment 26 was 198.28 amu. In order to calculate the number of thymidine residues in this fragment of the amplification product (and, by implication, the number of adenosine residues in the original target sequence), the mass increase (6735.64 amu−6537.36 amu=198.28 amu) is divided by the mass change due to changing from a dTTP to a bromo-dUTP (+64.97 amu). This results in a total of three bromo-uridine residues present in the (+) strand internal fragment 26 of the amplified product, a match to the expected count of three bromo-uridine residues (as would be predicted from the column in FIG. 8 entitled “Number of Alterable Ts”). Similarly, the mass increase of the (−) internal fragment was 520.41 amu (6973.63 amu−6453.22 amu). Again, this mass increase is divided by the mass change due to changing from a dTTP to a bromo-dUTP which results in a total of eight bromo-uridine residues present in the (−) strand internal fragment 28 of the amplified product, a match to the expected count of 8 bromo-uridine residues (as would be predicted from the column in FIG. 8 entitled “Number of Alterable Ts”). In an alternative embodiment, the samples are amplified separately with or without a mass-modified base, digested with enzyme, and analyzed at the same time.

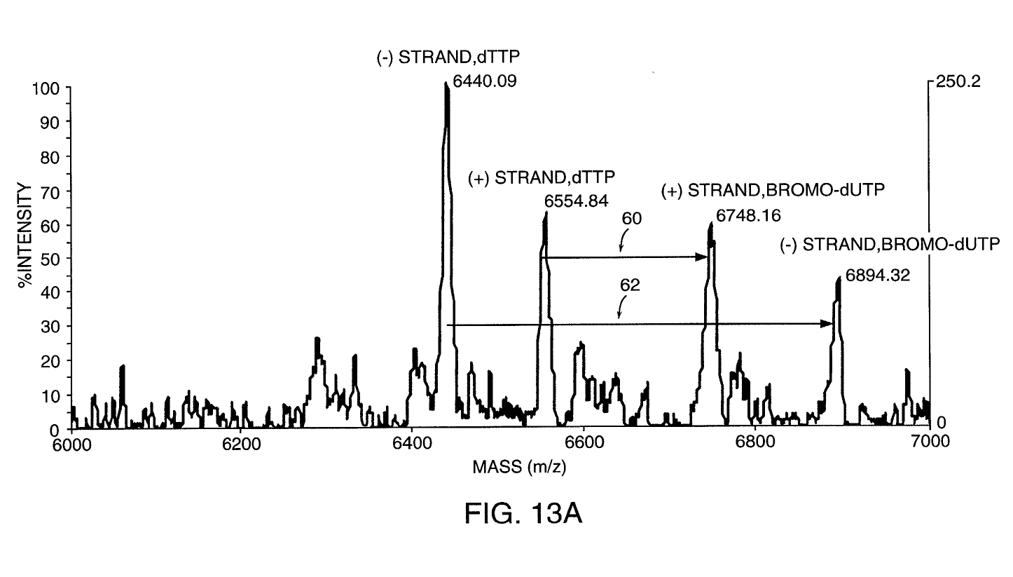

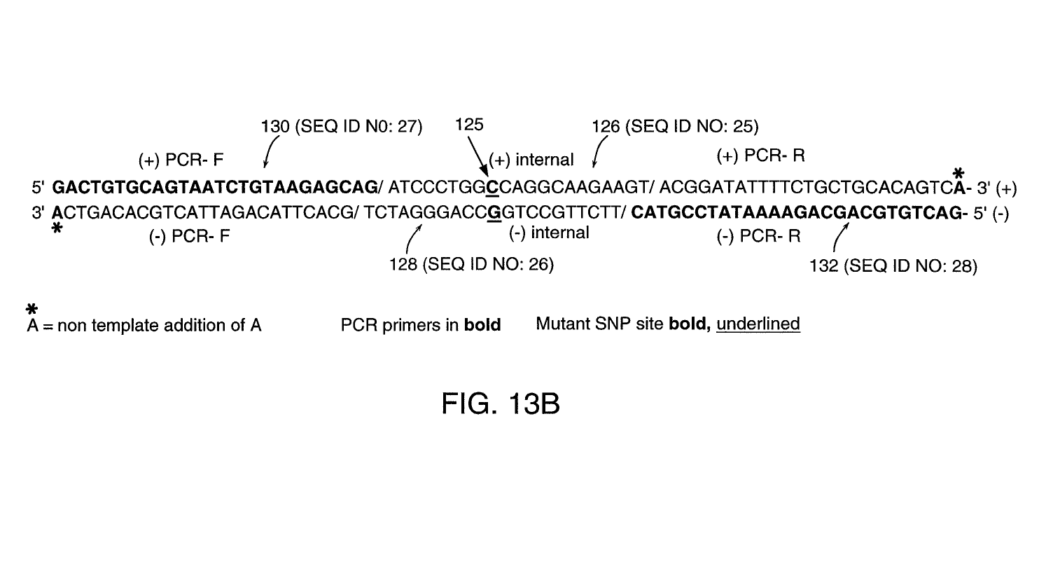

FIG. 13A shows a mass spectrograph when amplification reactions were performed in the presence of dTTP or bromo-dUTP and the amplification products were digested with an enzyme, as described above, and the two samples (incorporating either the dTTP or the bromo-dUTP) were mixed and analyzed in a single mass determination (rather than in separate mass determinations as in FIGS. 11 and 12). The analyzed sample is the same as that shown, for example, in FIG. 10, but has an adenosine residue to cytosine residue base change in the (+) strand. Accordingly, the (+) strand internal fragment 126 (SEQ ID NO: 25) has three alterable thymidine residues and the (−) strand internal fragment 128 (SEQ ID NO: 26) has seven alterable thymidine residues. The changed site 125 is shown in bold and underlined (FIG. 13B). The two PCR primers 130, 132 (SEQ ID NOS: 27 and 28, respectively) are shown in bold type. Arrow 60 depicts the mass peak shift for the (+) strand internal fragment 126 of the amplified product which was amplified in the absence of the mass-modified base in comparison to the (+) strand internal fragment 126 of the amplified product which was amplified in the presence of the mass-modified base. The mass shift was 193.32 amu (6748.16 amu−6554.84 amu). When 193.32 amu is divided by the mass change due to changing from a dTTP to a bromo-dUTP (+64.97 amu), a calculated total of three bromo-dUTP residues is shown to be present in the (+) strand internal fragment 126 of the amplified product, a match to the expected count of three bromo-dUTP residues. Similarly, arrow 62 depicts the mass peak shift for the (−) internal fragment 128 of the amplified product which was amplified in the absence of the mass-modified base in comparison to the (−) internal fragment 128 of the amplified product which was amplified in the presence of the mass-modified base. The mass shift was 454.23 amu (6894.32 amu−6440.09 amu). When 454.23 amu is divided by the mass change due to changing from a dTTP to a bromo-dUTP (+64.97 amu), a calculated total of seven bromo-uridine residues is shown to be present in the (−) strand internal fragment 128 of the amplified product, a match to the expected count of seven bromo-uridine residues. If these results are compared to those in FIGS. 11 and 12, it can be seen that the change from eight bromo-uridine residues in the (−) internal fragment in FIGS. 11 and 12 to seven bromo-uridine residues in the (−) internal fragment 128 in FIG. 13B indicates that the (+) internal fragment 126 of FIG. 13B has one fewer adenosine residue than does the (+) internal fragment 26 of FIGS. 11 and 12.

The experiment described above worked well when the sample was amplified with bromo-dUTP, but the restriction digestion step failed when the sample was amplified with bromo-dCTP, rather than bromo-dUTP. Possibly, modification of the restriction enzyme recognition site with bromine (in the negative strand only) can interfere with recognition by the enzyme. In this example, labeling with bromo-dUTP incorporates a single bromo-dUTP into the negative strand recognition sequence, which did not cause a problem. However, when the same protocol was attempted with bromo-dCTP, the new protocol introduced three bromo-dCTPs into the recognition sequence, and it was no longer recognized by the restriction endonuclease. Possibly, the increased number of bromines and/or the location of the cytidine residues in a more critical region than the uridine residues caused the less than optimal result.

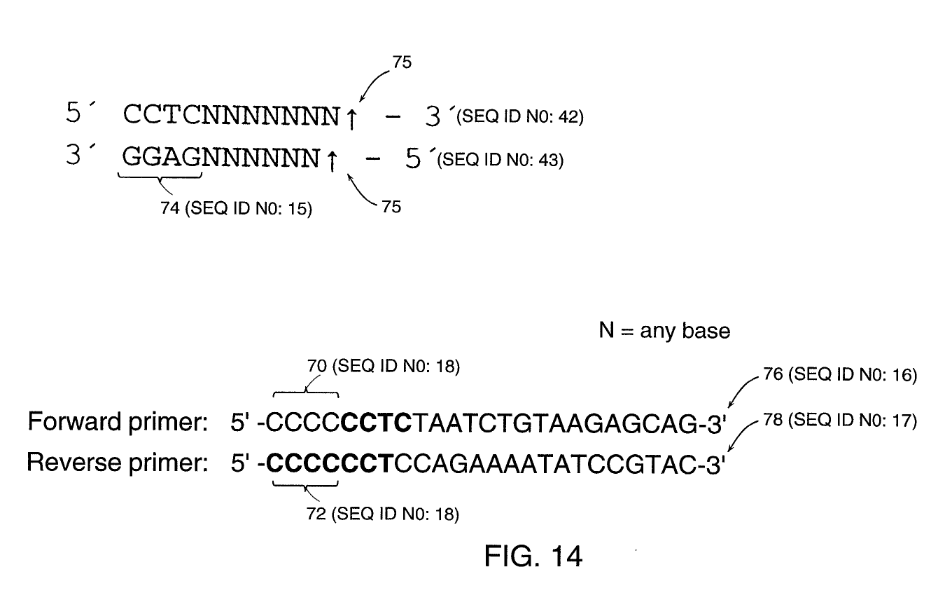

One solution was to use a type IIs restriction endonuclease having a recognition sequence that does not contain cytidine or uridine residues in at least one strand of the recognition sequence. Mnl I (Catalog#R0163, available from New England BioLabs, Beverley, Mass.), which has a four base recognition sequence 74 of 3′-GGAG-5′ (SEQ ID NO: 15) in the minus strand, was identified. The forward and reverse primers described above were redesigned with a Mnl I recognition sequence 76, 78 (SEQ ID NOS: 16 and 17, respectively) (FIG. 14 ). Cytidine and thymidine residues were used in the primers such that only guanosine and adenosine residues could be incorporated into the complementary strand. Again, four additional 5′ residues were added to the primer as a cap sequence because it is thought that some restriction enzymes require a few 5′ residues next to the restriction site for full activity. A sequence of -CCCC- (SEQ ID NO: 18) was selected for the cap sequence 70, 72 so that the complementary strand would contain only guanosine residues (and no cytidine or uridine residues). With this primer design, the entire restriction enzyme recognition sequence does not incorporate any mass-modified bases in either strand. Primer designs also can consider whether or not to have mass-modified modified bases incorporated in the region outside of the recognition site itself in order to optimize results. In some embodiments with type IIs restriction enzymes, it may not be important whether the amplified DNA has mass-modified residues at the site of cleavage in contrast to the recognition site.

Primers 76, 78 were used to amplify the same sequence as shown in FIG. 10 by the method described above. The resultant amplification products were equally well digested with Mnl I whether the amplification product was amplified with unmodified dNTPs, with bromo- or iodo-modified dUTP, or with bromo- or iodo-modified dCTP. Thus, the potential problem of mass-modified bases interfering with restriction digestion can be prevented. As shown in FIG. 14 by the arrows 75, Mnl I produces a staggered cut at six bases on one strand (SEQ ID NO: 43) and at seven bases on the complementary strand (SEQ ID NO: 42) from the recognition sequence, in contrast to the recognition sequence of Bsg I which produces a staggered cut sixteen bases from the recognition sequence. Thus, Bsg I removes more of a primer sequence than does Mnl I. Alternatively, some enzymes may tolerate various mass-modifiers and such primer design steps may not be necessary.

A PCR primer also can be removed by incorporation of a cleavable residue at or near the 3′ end of the primer, such as an RNA residue or other chemically cleavable residues. For example, an RNase digestion or digestion with sodium hydroxide at elevated temperature can remove the primer, or a uridine residue can be cleaved with uracil-N-glycosylase. Also, a double-stranded amplification product can be separated into two separate strands and then a segment of the strand can be removed with a restriction endonuclease that acts on a single strand. Also, groups that protect against exonuclease digestion (for example, phosphorothioates) can be incorporated into a PCR primer. At the appropriate time, some of that sequence can removed during a digestion step. Additionally, sites capable of blocking 5′ to 3′ digestion such as digestion with T7 gene 6 endonuclease can be used. Other methods to remove a primer can be used as well.

E. Mass-Tuning and Visualization Techniques

PCR products usually contain plus and minus strands of identical length. These strands may be significantly different in mass and resolvable. Alternatively, the strands may be relatively close in mass and are not able to be resolved in a satisfactory manner. Rarely, the strands are of exactly the same mass. If the target sequence to be examined is known, whether or not it is anticipated that the strands will be satisfactorily resolvable can be determined in advance. In the case where the strands of the PCR product, or fragments thereof, are relatively close in mass, and therefore not resolved in a satisfactory manner, it may be difficult to assign accurate mass values. However, the mass accuracy can be improved by ensuring that the two strands of the PCR product, or fragments thereof, have significantly different masses. Many techniques are suitable to alter the mass of the amplified products (or to “mass tune” the strands).

In one example, a 5′ non-base residue (or residues) can be added to one PCR primer. A non-base residue adds mass to the primer and the PCR product strand to which they are attached. Because they cannot be copied, there is no additional mass added to the complement of the primer sequence. For example, spacer arms, C18 groups, biotin, or a 3′ or 5′ phosphate, can be used to add mass to only one strand of PCR product. Commercially available C 18 groups can be incorporated into PCR products and result in about a 250 amu increase in the mass of the strand for each C18 incorporated. In another example, sequences are added to the 5′ end of the primer, which either induce or prevent 3′ template-independent base addition by Taq DNA polymerase. See. e.g., Brownstein et al. (1998), BioTechniques 20:1004-10; Magnuson et al. (1998) BioTechniques 21:700-9, both of which are incorporated by reference herein. The design can induce or prevent the addition of a single adenosine residue to the desired PCR strand, altering the mass by 313 amu. The length of the two strands can be adjusted by designing the PCR so that one strand has an extra A residue and the other does not. As shown in FIGS. 8 and 10 (by a bold “A” or an asterisk adjacent an “A”), the primers from that example promoted non-template addition of an adenosine residue.

In another example, poorly resolvable strands can be analyzed by isolating one of the strands, or a fragment thereof, as known in the art. Strands also can be isolated for other reasons of convenience even if they are adequately resolvable. In one example, one strand of a PCR product can be digested, for example, with lambda exonuclease I. In another example, only one strand of a PCR product is recovered, for example, by adding 5′ biotin to one primer and isolating it with immobilized streptavidin. The captured nucleic acid also can be a duplex with the non-biotinylated strand being eluted for further analysis. Other methods of chromatographic isolation also can be used.

In another example, asymmetric PCR (primer extension with a single primer and dNTPs) is used so that only one strand is copied. See, e.g., Jurinke et al. (1998), Rapid Communications in Mass Spectrometry 12:50-2, which is incorporated by reference herein. Thus, a single strand of PCR product can be analyzed rather than both strands. This strategy can be accomplished by conducting the PCR in two stages. First, PCR is conducted with two PCR primers. Second, asymmetric PCR is conducted in the presence and absence of mass-modified dNTPs but with only one primer. The second primer can be one of the PCR primers or a primer internal to the first set of PCR primers. The primer can be designed to copy any desired segment, for example, to further localize a mutation site.

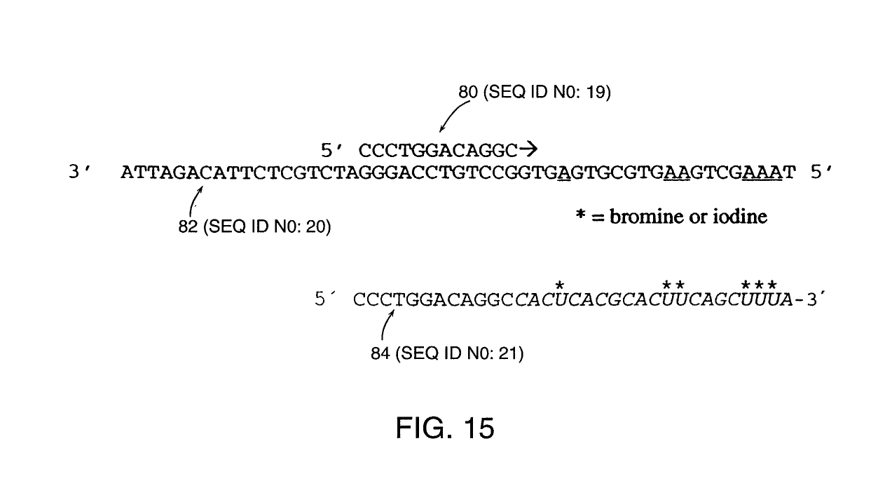

As shown in FIG. 15, a 12-mer primer 80 (SEQ ID NO: 19) was designed to extend to the end of a target DNA sequence 82 (SEQ ID NO: 20), producing a twenty-one base extension product 84 (SEQ ID NO: 21). The terms “extension” and “amplification” (and other grammatical versions of both) are used interchangeably in this context because an extension reaction is one form of an amplification reaction (for example, asymmetric PCR). The primer 80 was extended in the presence of bromo-dUTP or iodo-dUTP and the extension product incorporated the mass-modified base. The total length of the primer extension product is twenty-one bases and it incorporates six bromo-dUTP bases or six iodo-dUTP bases (depending upon which mass-modified base was used).

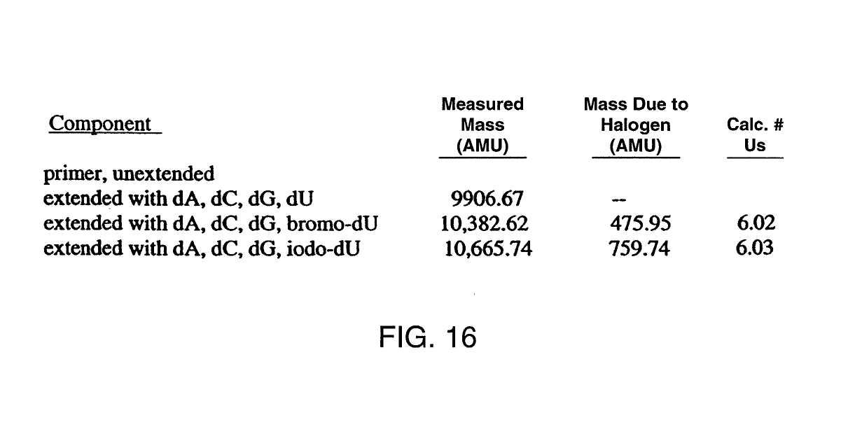

As shown in FIG. 16, the extension product obtained from extension in the absence of mass-modified bases has a mass (9906.67 amu) that is less than the mass from the extension products obtained from extensions that incorporate either bromo-dUTP or iodo-dUTP (10382.62 amu and 10665.74 amu, respectively). This difference in mass between the extension product extended without the mass-modified base and the extension products extended with the mass-modified bases (475.95 amu and 759.74 amu for bromo-dUTP and iodo-dUTP, respectively) is divided by the mass increase due to the mass-modified base (about 78.9 amu for bromine and about 125.9 amu for iodine), which results in the correct calculation of the incorporation of six bromo-dUTPs or six iodo-dUTPs.

In another example, poorly resolvable strands are examined by removing a segment from one of the strands, or a fragment thereof. For example, groups that protect against exonuclease digestion (for example, phosphorothioates) can be incorporated into a PCR primer and later, some of that sequence is removed during a digestion step. Another example is to add a cleavable group to one PCR primer, such as a uridine residue (cleavable with uracil-N-glycosylase) or an RNA residue cleavable by a basic solution. Another example is to cleave the PCR product with a Type IIs restriction enzyme, but only at one of the PCR primers. Assuming that a Type IIs enzyme such as BglI is used, this strategy can produce strands differing in length by two bases. Four fragments will be generated. Each fragment will differ in length from its complement by two bases due to the staggered cuts of BglI, as described above. The same result can be obtained using a recognition site for a Type IIs restriction enzyme that produces staggered ends in one primer and using a restriction site or recognition site in the second PCR primer for a restriction enzyme that produces blunt ends.

F. Additional Embodiments

In some situations, such as single base SNP analysis, PCR may be performed with only a single base between the primers. If the primers are relatively short, restriction enzyme digestion may not be necessary. However, if restriction enzymes are used to remove uninformative sequence, the primers can be designed with a restriction site such that the base at the analyzed SNP site stays with one of the PCR primers. In either manner, the technique would confirm the mutation at an exact position and is suitable for SNP analysis. In some scenarios, this assay could be less expensive and simpler than other assays in the art.

Because large mass differences can be obtained between a mutant and a wild type template in this assay, it may be possible to obtain accurate quantitative data for the proportions of two alleles using populations of targets rather than single targets.

In another embodiment, similar to the primer extension method described above, primer extensions can be carried out on a target with a mixture of dNTPs and ddNTPs (dideoxynucleotides). The extensions are conducted in the presence of ddNTPs of a single base type which is different from the mass-modified base that is used. Extensions with a combination of unmodified dNTPs, a mass-modified base, and a ddNTP of a type different from the mass-modified base produce extension ladders terminating at positions defined by the ddNTP base. The incorporation of one or more mass-modified bases between any two termination positions is detected as an increased mass compared to a ladder made with no mass-modified bases. The mutation will be located between the first 3′ termination position after the primer sequence with the increased mass, and the previous termination site. Multiple mutations can be detected by sequential analysis at each termination site.

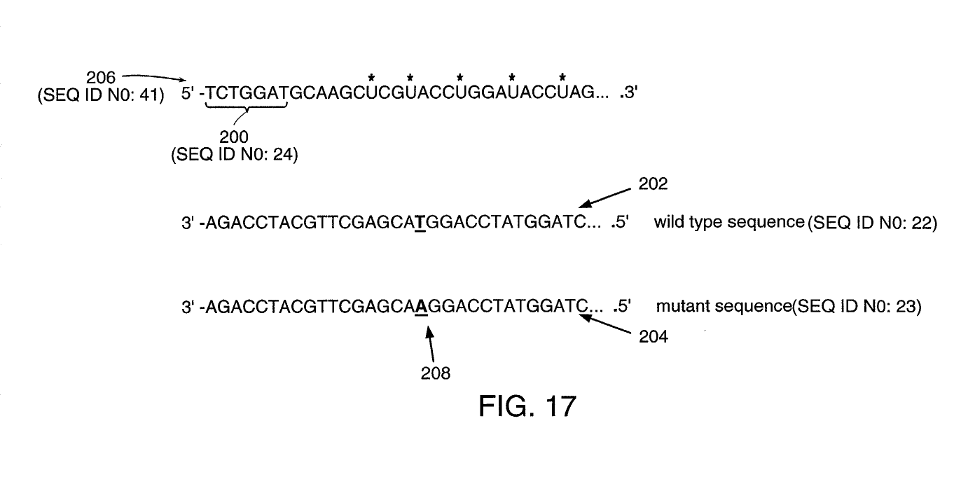

Referring to FIGS. 17, 18, and 19, the technique typically employs two unmodified dNTPs, a mixture of one unmodified dNTP and one ddNTP of the same base type, and one mass-modified dNTP. In this example, a wild type sequence 202 (SEQ ID NO: 22) and a mutant of that sequence 204 (SEQ ID NO: 23) with a T to A mutation 208 are used. A primer 200 (SEQ ID NO: 24) is extended with a mixture of dATP, dCTP, bromo-dUTP, and a mixture of dGTP and ddGTP. Bromo-uridine residues are indicated by an asterisk.

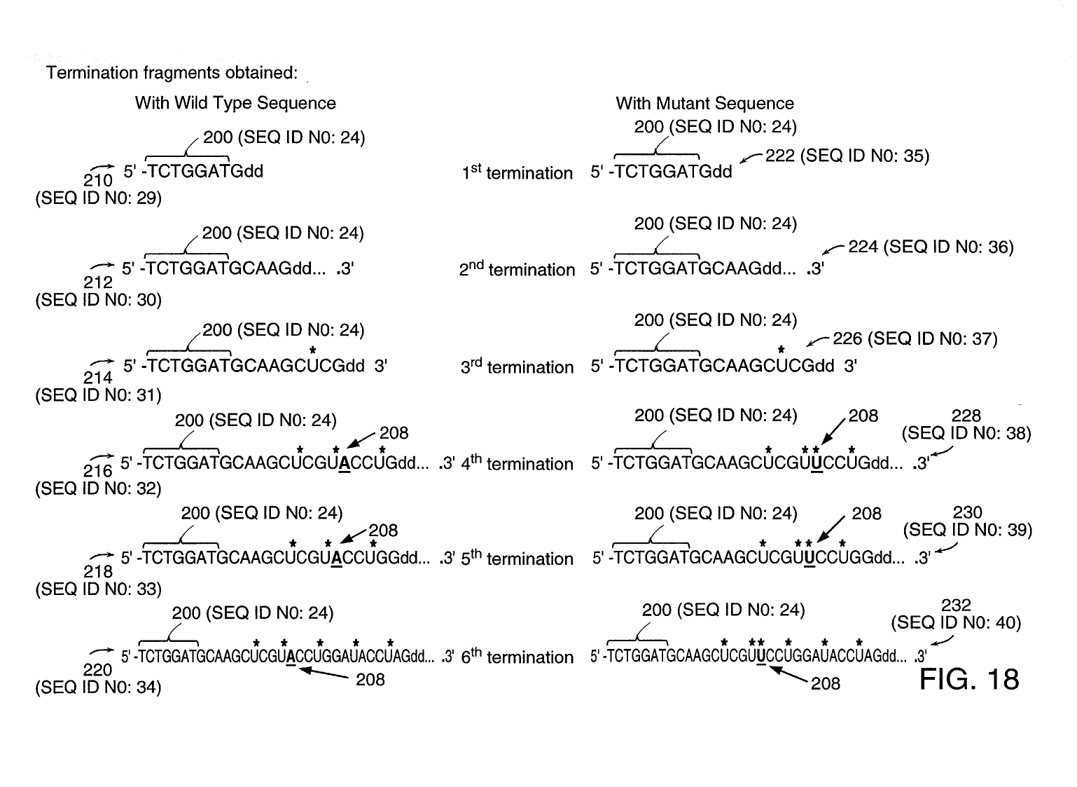

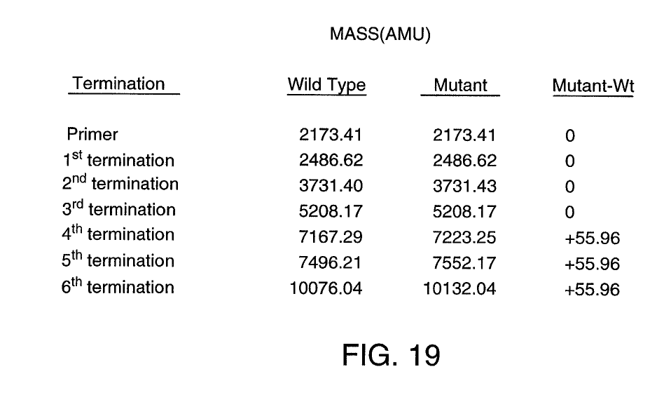

Under these conditions, a ladder of sequences with terminations corresponding to the positions of cytidine residues in the target sequences 202, 204 is obtained. The longest ladder sequence 206 (SEQ ID NO: 41), with the cytidine residue positions is shown in FIG. 17. The termination fragments 210, 212, 214 , 216, 218, 220 (SEQ ID NOS: 29-34, respectively) of the extended wild type nucleic acid and the termination fragments 222, 224, 226, 228, 230, 232 (SEQ ID NOS: 35-40, respectively) of the extended mutant nucleic acid are shown in FIG. 18. As shown in FIG. 19, for the first three termination fragments (numerals 210, 212, 214 for wild type and numerals 222, 224, 226 for mutant) there is no difference in mass between the primer extensions from wild type and mutant template. This means there were no mutations discovered with bromo-dUTP as the mass-modifier (no change in the number of adenosine residues in the target sequences 202, 204 up to the position of the termination in third termination fragments 214 , 226). In the fourth termination fragments 216, 228, there was a +55.96 amu increase in the mass of the termination fragment 228 for the mutant template as compared with the wild type template. A mass change of +55.96 amu is indicative of incorporation of a bromo-dUTP instead of dATP (see FIG. 3) and correlates with a T to A base change between the wild type and the mutant target. Because other base changes lead to only slightly different net mass changes (for example incorporation of a bromo-dUTP instead of a dGTP changes mass by 39.96 amu), it may be difficult to unambiguously identify the exact allelic pair in the mutant and wild type (depending upon how much resolution is generated by the mass spectrometer). The mass increase would be even greater and easier to detect by incorporation of an iodo-dUTP residue (+102.86 amu). The exact location of the mutation is not disclosed (unless the wild type sequence is known), but it falls between the third and fourth termination sequences 214 , 226, 216, 228 (always ending with ddGTP). There are no additional mutations disclosed between the fifth and sixth termination sequences 218, 230, 220, 232 because there is no further mass change.

Two variations of this method should be noted. First, if the wild type sequence is known, the masses of the sequence ladders for wild type can be calculated, obviating the need to actually prepare the sequence ladder from the reference specimen. Second, the technique also can be conducted with the embodiment utilizing four amplification reactions. This may be desirable when the sequence is unknown. In this case, the number of bases of any one type in each termination fragment (for example, adenosine residues in the target sequence generating incorporation of dUTP or bromo-dUTP in the termination fragment) can be determined by generating the sequence ladder separately with dUTP and bromo-dUTP, for example. The number of bromo-dUTP residues incorporated into each fragment is equal to the difference in masses of the fragments (extended in the presence of dUTP or bromo-dUTP) divided by 78.9 amu.

For many types of analysis, the wild type sequence is known, which assists in designing primers and predicting masses. At a minimum, enough of the sequence should be known to design PCR primers. However, completely unknown sequences can be analyzed for mutations by comparing different amplification products by using inverse PCR. In inverse PCR, primers may span a completely unknown sequence, typically formed by ligating a circular template. Generally, a plasmid (or other vector) is linearized, and an unknown sequence of nucleic acid is ligated into the plasmid which is then re-circularized. Then, PCR is conducted with primers complementary to the known sequences in the plasmid that now flank the unknown sequence. Such amplification is done with or without mass-modified bases, depending upon which embodiment of the invention is practiced.

Also, RNA can be analyzed by converting it to DNA using a reverse transcriptase in the presence of mass-modified bases, especially if the reverse transcriptases will efficiently incorporate mass-modified bases. This situation would be an amplification. RNA also can be analyzed by converting it to DNA with regular bases and reverse transcriptase, and then further amplifying the DNA by regular PCR with mass-modified bases.

Additionally, a single strand of DNA can be transcribed into a single strand of RNA by RNA transcription with primers containing T7, T3, or SP6 promoter sequences. Typically, a PCR primer containing a promoter sequence is used to amplify target DNA. In one embodiment, the promoter sequence typically is about 23 bases long and initiates synthesis about 17 bases from the 5′ end of the promoter. The amplified DNA is copied by making a complementary copy of single stranded RNA using rATP, rCTP, rGTP, and rUTP. This situation produces even higher amplification and also can permit analysis of one DNA strand at a time (if the DNA is double-stranded). For example, each primer in an amplification reaction could have a different RNA polymerase recognition site such that each strand could be transcribed independently of the other. RNA products also are somewhat more stable in the mass spectrometer than DNA, so longer lengths can be read. Mass-modified rNTPs (for example, bromo-rUTP or bromo-rCTP), such as halogen-modified rNTPs, may be efficiently incorporated. For example, ribose-modified 2′ fluoro and 2′ amino deoxynucleoside triphosphates can be incorporated by T7 RNA polymerase and stabilizes RNA product, although the incorporation is not extremely efficient. The techniques described above can be accomplished with these unmodified and mass-modified rNTPs in amplification reactions. Thus, by using amplification reactions utilizing rNTPs and *rNTPs (for example, instead of, or in addition to, PCR), one can carry out the amplification techniques described above to count the number of bases in a strand of nucleic acid or to identify a changed base between two nucleic acids.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

Each of the patent documents and scientific publications disclosed hereinabove is incorporated by reference herein.

What is claimed is:

1. A method for determining the number of mass-modified nucleobases incorporated in an amplified nucleic acid, the method comprising the steps of:
amplifying a sample comprising a target nucleic acid in the presence of a mass-modified nucleobase to produce an amplified product incorporating the mass-modified nucleobase, wherein the mass-modified nucleobase has a mass more than about 27 amu greater than the mass of the corresponding unmodified nucleobase;
amplifying a second sample comprising the target nucleic acid in the absence of mass-modified nucleobases to produce a reference nucleic acid;
comparing the mass of one strand of the amplified product with the mass of one strand of the reference nucleic acid; and
determining the number of mass-modified nucleobases incorporated in the one stand of the amplified product.

2. The method of claim 1 wherein the mass-modified nucleobase comprises a halogen.

3. The method of claim 1 wherein the amplified product comprises two complementary strands.

4. The method of claim 1 wherein the mass-modified nucleobase excludes isotopic variants of the elemental constituents of the base.

5. The method of claim 1 wherein the mass-modified nucleobase is selected from the group consisting of 5-Bromo-2′-deoxycytidine-5′-triphosphate, 5-Iodo-2′-deoxycytidine-5′-Triphosphate, 5-Iodo-2′-deoxyuridine-5′-triphosphate, 5-Bromo-2′-deoxyuridine-5′-Triphosphate, 5-iodocytidine-5′-Triphosphate, 5-Iodouridine-5′-Triphosphate, 5-bromocytidine-5′-Triphosphate, and 5-bromouridine-5′-Triphosphate.

6. The method of claim 1 wherein the amplified product comprises RNA.

7. The method of claim 3 further comprising the steps of performing the comparing and determining steps on a second strand of the amplified product which is complementary to the one strand of amplified product, and on a second strand of the reference nucleic acid which is complementary to the one strand of the reference nucleic acid.

8. The method of claim 3 further comprising the step of isolating the one strand of the amplified product.

9. The method of claim 8 wherein the isolating step comprises amplifying by asymmetric PCR the one strand of the amplified product, degrading a second strand of the amplified product which is complementary to the one strand of the amplified product, capturing of the one strand of the amplified product, or chromatographically isolating the one strand of the amplified product, or a combination thereof.

10. The method of claim 3 further comprising the step of modifying the mass of the one strand of the amplified product.

11. The method of claim 10 wherein the modifying step comprises amplifying the target nucleic acid with at least one primer comprising a non-base residue, promoting non-template addition of a base, inducing template-independent base addition by a DNA polymerase, or preventing template-independent base addition by a DNA polymerase, or a combination thereof.

12. The method of claim 1 further comprising the step of placing the target nucleic acid in a plasmid.

13. The method of claim 1 further comprising the step of reverse transcribing a molecule of RNA into a molecule of DNA to form the target nucleic acid.

14. The method of claim 1 further comprising the step of using mass spectrometry to determine the masses of the one strand of the amplified product and the one strand of the reference nucleic acid.

15. The method of claim 1 wherein two primers are used to amplify the target nucleic acid, the method further comprising the step of subtracting the number of mass-modified nucleobases incorporated in the one strand of the amplified product at a locus complementary to one of the primers from the total number of mass-modified nucleobases incorporated in the one strand of the amplified product.

16. A method for determining the number of mass-modified nucleobases incorporated in an amplified nucleic acid, the method comprising the steps of:
amplifying a sample comprising a target nucleic acid in the presence of a mass-modified nucleobase to produce an amplified product incorporating the mass-modified nucleobase, wherein the mass-modified nucleobase has a mass more than about 27 amu greater than the mass of the corresponding unmodified nucleobase;
amplifying a second sample comprising the target nucleic acid in the absence of mass-modified nucleobases to produce a reference nucleic acid;
removing a segment from the amplified product to form a shortened amplified product;
removing the segment from the reference nucleic acid to form a shortened reference nucleic acid;
comparing the mass of one strand of the shortened amplified product with the mass of one strand of the shortened reference nucleic acid; and
determining the number of mass-modified nucleobases incorporated in the one strand of the shortened amplified product.

17. The method of claim 16 wherein a primer used to amplify the target nucleic acid comprises an enzyme recognition site.

18. The method of claim 16 wherein a primer used to amplify the target nucleic acid comprises a group which protects against nucleic acid degradation.

19. A method for determining a base change in a nucleic acid, the method comprising the steps of:
amplifying a sample comprising a target nucleic acid in the presence of a mass-modified nucleobase to produce an amplified product incorporating the mass-modified nucleobase;
comparing the mass of one strand of the amplified product with the mass of one strand of a reference nucleic acid incorporating the mass-modified nucleobase; and
determining the identity of a base responsible for a base composition difference, if any, between the amplified product and the reference nucleic acid.

20. The method of claim 19 further comprising the step of amplifying a second sample comprising a nucleic acid in the presence of the mass-modified nucleobase to produce the reference nucleic acid.

21. The method of claim 19 wherein the amplified product comprises two complementary strands.

22. The method of claim 19 wherein the mass-modified nucleobase has a mass more than about 27 amu greater than the mass of the corresponding unmodified base.

23. The method of claim 19 wherein the mass-modified nucleobase excludes isotopic variants of the elemental constituents of the base.

24. The method of claim 19 wherein the mass-modified nucleobase is selected from the group consisting of 5-Bromo-2′-deoxycytidine-5′-triphosphate, 5-Iodo-2′-deoxycytidine-5′-Triphosphate, 5-Iodo-2′-deoxyuridine-5′-triphosphate, 5-Bromo-2′-deoxyuridine-5′-Triphosphate, 2-Thiothymidine-5′-triphosphate, 5-iodocytidine-5′-Triphosphate, 5-Iodouridine-5′-Triphosphate, 2-thiouridine-5′-Triphosphate, 4-thiouridine-5′-triphosphate, 2-thiocytidine-5′-Triphosphate, 5-bromocytidine-5′-Triphosphate, and 5-bromouridine-5′-Triphosphate.

25. The method of claim 19 wherein the amplified product comprises RNA.

26. The method of claim 21 further comprising the step of performing the comparing step on a second strand of the amplified product which is complementary to the one strand of amplified product, and on a second strand of the reference nucleic acid which is complementary to the one strand of the reference nucleic acid.

27. The method of claim 21 further comprising the step of isolating the one strand of the amplified product.

28. The method of claim 27 wherein the isolating step comprises amplifying by asymmetric PCR the one strand of the amplified product, degrading a second strand of the amplified product which is complementary to the one strand of the amplified product, capturing the one strand of the amplified product, or chromatographically isolating the one strand of the amplified product, or a combination thereof.

29. The method of claim 21 further comprising the step of modifying the mass of the one strand of the amplified product.

30. The method of claim 29 wherein the modifying step comprises amplifying the target nucleic acid with at least one primer comprising a non-base residue, promoting non-template addition of a base, inducing template-independent base addition by a DNA polymerase, or preventing template-independent base addition by a DNA polymerase, or a combination thereof.

31. The method of claim 19 further comprising the step of placing the target nucleic acid in a plasmid.

32. The method of claim 19 further comprising the step of reverse transcribing a molecule of RNA into a molecule of DNA to form the target nucleic acid.

33. The method of claim 19 further comprising the step of using mass spectrometry to determine the masses of the one strand of the amplified product and the one strand of the reference nucleic acid.

34. The method of claim 19 wherein the mass-modified nucleobase comprises a halogen.

35. A method for determining a base change in a nucleic acid, the method comprising the steps of:
amplifying a sample comprising a target nucleic acid in the presence of a mass-modified nucleobase to produce an amplified product incorporating the mass-modified nucleobase;
removing a segment from the amplified product to form a shortened amplified product;
comparing the mass of one strand of the shortened amplified product with the mass of one strand of a reference nucleic acid incorporating the mass-modified nucleobase; and
determining the identity of a base responsible for a base composition difference, if any, between the shortened amplified product and the reference nucleic acid.

36. The method of claim 35 wherein a primer used to amplify the target nucleic acid comprises an enzyme recognition site.

37. The method of claim 35 wherein a primer used to amplify the target nucleic acid comprises a group which protects against nucleic acid degradation.

38. A method for analyzing the base composition of a nucleic acid comprising the steps of:
comparing the mass of a first nucleic acid incorporating a mass-modified nucleobase with the mass of a second nucleic acid;
comparing the mass difference, if any, between the first nucleic acid and the second nucleic acid with a matrix of possible mass differences between the first nucleic acid and the second nucleic acid; and
determining from the matrix the identity of a base responsible for a base composition difference, if any, between the first nucleic acid and the second nucleic acid.

39. The method of claim 35 wherein the second nucleic acid incorporates the mass-modified nucleobase.


SEQUENCE LISTING (43 sequences)

SEQ ID NO: 1 - DNA, 12 bp, Artificial Sequence
  forward primer
taatctgtaaga

SEQ ID NO: 2 - DNA, 51 bp, Artificial Sequence
  target strand
cagaaaatatccgtacttctcgcctgtccagggatctgctcttacagatta

SEQ ID NO: 3 - DNA, 51 bp, Artificial Sequence
  amplification product
taatctgtaagagcagancccnggncaggcgagaagnacgganannnncng

SEQ ID NO: 4 - DNA, 51 bp, Artificial Sequence; identical to SEQ ID NO: 3
  amplified product

SEQ ID NO: 5 - DNA, 51 bp, Artificial Sequence
  mutant target sequence
cagaaaatatccgtgcttctcgcctgtccagggatctgctcttacagatta

SEQ ID NO: 6 - DNA, 51 bp, Artificial Sequence
  reference nucleic acid
taatctgtaagagcagancccnggncaggcgagaagcacgganannnncng

SEQ ID NO: 7 - DNA, 51 bp, Artificial Sequence; identical to SEQ ID NO: 6
  amplified product

SEQ ID NO: 8 - DNA, 6 bp, Artificial Sequence
  recognition sequence
gtgcag

SEQ ID NO: 9 - DNA, 26 bp, Artificial Sequence
  primer
gactgtgcagtaatctgtaagagcag

SEQ ID NO: 10 - DNA, 26 bp, Artificial Sequence
  primer
gactgtgcagcagaaaatatccgtac

SEQ ID NO: 11 - DNA, 25 bp, Artificial Sequence
  primer complement
gcacttacagattactgcacagtca

SEQ ID NO: 12 - DNA, 25 bp, Artificial Sequence
  primer complement
acggatattttctgctgcacagtca

SEQ ID NO: 13 - DNA, 21 bp, Artificial Sequence
  shortened amplification product
atccctggacaggcaagaagt

SEQ ID NO: 14 - DNA, 21 bp, Artificial Sequence
  shortened amplification product
ttcttgcctgtccagggatct

SEQ ID NO: 15 - DNA, 4 bp, Artificial Sequence
  recognition sequence
gagg

SEQ ID NO: 16 - DNA, 24 bp, Artificial Sequence
  forward primer
cccccctctaatctgtaagagcag

SEQ ID NO: 17 - DNA, 24 bp, Artificial Sequence
  reverse primer
cccccctccagaaaatatccgtac

SEQ ID NO: 18 - DNA, 4 bp, Artificial Sequence
  cap sequence
cccc

SEQ ID NO: 19 - DNA, 12 bp, Artificial Sequence
  primer
ccctggacaggc

SEQ ID NO: 20 - DNA, 51 bp, Artificial Sequence
  target sequence
taaagctgaagtgcgtgagtggcctgtccagggatctgctcttacagatta

SEQ ID NO: 21 - DNA, 33 bp, Artificial Sequence
  extension product
ccctggacaggccacncacgcacnncagcnnna

SEQ ID NO: 22 - DNA, 31 bp, Artificial Sequence
  wild type sequence
ctaggtatccaggtacgagcttgcatccaga

SEQ ID NO: 23 - DNA, 31 bp, Artificial Sequence
  mutant sequence
ctaggtatccaggaacgagcttgcatccaga

SEQ ID NO: 24 - DNA, 7 bp, Artificial Sequence
  primer
tctggat

SEQ ID NO: 25 - DNA, 21 bp, Artificial Sequence
  internal fragment
atccctggccaggcaagaagt

SEQ ID NO: 26 - DNA, 21 bp, Artificial Sequence
  internal fragment
ttcttgcctggccagggatct

SEQ ID NO: 27 - DNA, 26 bp, Artificial Sequence
  primer
gactgtgcagtaatctgtaagagcag

SEQ ID NO: 28 - DNA, 26 bp, Artificial Sequence
  primer
gactgtgcagcagaaaatatccgtac

SEQ ID NO: 29 - DNA, 8 bp, Artificial Sequence
  wild type termination fragment
tctggatn

SEQ ID NO: 30 - DNA, 12 bp, Artificial Sequence
  wild type termination fragment
tctggatgcaan

SEQ ID NO: 31 - DNA, 16 bp, Artificial Sequence
  wild type termination fragment
tctggatgcaagcncn

SEQ ID NO: 32 - DNA, 22 bp, Artificial Sequence
  wild type termination fragment
tctggatgcaagcncgnaccnn

SEQ ID NO: 33 - DNA, 23 bp, Artificial Sequence
  wild type termination fragment
tctggatgcaagcncgnaccngn

SEQ ID NO: 34 - DNA, 31 bp, Artificial Sequence
  wild type termination fragment
tctggatgcaagcncgnaccngganaccnan

SEQ ID NO: 35 - DNA, 8 bp, Artificial Sequence
  mutant termination fragment
tctggatn

SEQ ID NO: 36 - DNA, 12 bp, Artificial Sequence
  mutant termination fragment
tctggatgcaan

SEQ ID NO: 37 - DNA, 16 bp, Artificial Sequence
  mutant termination fragment
tctggatgcaagcncn

SEQ ID NO: 38 - DNA, 22 bp, Artificial Sequence
  mutant termination fragment
tctggatgcaagcncgnnccnn

SEQ ID NO: 39 - DNA, 23 bp, Artificial Sequence
  mutant termination fragment
tctggatgcaagcncgnnccngn

SEQ ID NO: 40 - DNA, 31 bp, Artificial Sequence
  mutant termination fragment
tctggatgcaagcncgnnccngganaccnan

SEQ ID NO: 41 - DNA, 31 bp, Artificial Sequence
  longest ladder sequence
tctggatgcaagcncgnaccngganaccnag

SEQ ID NO: 42 - DNA, 11 bp, Artificial Sequence
  restriction fragment
cctcnnnnnnn

SEQ ID NO: 43 - DNA, 10 bp, Artificial Sequence
  restriction fragment
nnnnnngagg